SEQUENCE LISTING (38 sequences)

SEQ ID NO: 1 - protein, 119 aa, Artificial Sequence [sequence omitted]

SEQ ID NO: 2 - protein, 114 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 3 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 10 aa, Artificial Sequence
  Description of Artificial Sequence Synthetic peptide
GYTFTSYWLH

SEQ ID NO: 6 - protein, 18 aa, Artificial Sequence
  Description of Artificial Sequence Synthetic peptide
GMIDPSNSDTRFNPNFKD

SEQ ID NO: 7 - protein, 12 aa, Artificial Sequence
  Description of Artificial Sequence Synthetic peptide
ATYRSYVTPLDY

SEQ ID NO: 8 - protein, 17 aa, Artificial Sequence
  Description of Artificial Sequence Synthetic peptide
KSSQSLLYTSSQKNYLA

SEQ ID NO: 9 - protein, 7 aa, Artificial Sequence
  Description of Artificial Sequence Synthetic peptide
WASTRES

SEQ ID NO: 10 - protein, 9 aa, Artificial Sequence
  Description of Artificial Sequence Synthetic peptide
QQYYAYPWT

SEQ ID NO: 11 - protein, 25 aa, Artificial Sequence [sequence omitted]

SEQ ID NO: 12 - protein, 13 aa, Artificial Sequence
  Description of Artificial Sequence Synthetic peptide
WVRQAPGKGLEWV

SEQ ID NO: 13 - protein, 30 aa, Artificial Sequence
  Description of Artificial Sequence Synthetic polypeptide
RFTISADTSKNTAYLQMNSLRAEDTAVYYC

SEQ ID NO: 14 - protein, 11 aa, Artificial Sequence
  Description of Artificial Sequence Synthetic peptide
WGQGTLVTVSS

SEQ ID NO: 15 - protein, 23 aa, Artificial Sequence
  Description of Artificial Sequence Synthetic peptide
DIQMTQSPSSLSASVGDRVTITC

SEQ ID NO: 16 - protein, 15 aa, Artificial Sequence
  Description of Artificial Sequence Synthetic peptide
WYQQKPGKAPKLLIY

SEQ ID NO: 17 - protein, 32 aa, Artificial Sequence
  Description of Artificial Sequence Synthetic polypeptide
GVPSRFSGSGSGTDFTLTISSLQPEDFATYYC

SEQ ID NO: 18 - protein, 11 aa, Artificial Sequence
  Description of Artificial Sequence Synthetic peptide
FGQGTKVEIKR

SEQ ID NO: 19 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 21 - protein, 449 aa, Artificial Sequence
  Description of Artificial Sequence Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 220 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 17 aa, Artificial Sequence
  Description of Artificial Sequence Synthetic peptide
KSSQSLLYTSSQKNYLA

SEQ ID NO: 24 - protein, 7 aa, Artificial Sequence
  Description of Artificial Sequence Synthetic peptide
WASTRES

SEQ ID NO: 25 - protein, 9 aa, Artificial Sequence
  Description of Artificial Sequence Synthetic peptide
QQYYAYPWT

SEQ ID NO: 26 - protein, 10 aa, Artificial Sequence
  Description of Artificial Sequence Synthetic peptide
GYTFTSYWLH

SEQ ID NO: 27 - protein, 18 aa, Artificial Sequence
  Description of Artificial Sequence Synthetic peptide
GMIDPSNSDTRFNPNFKD

SEQ ID NO: 28 - protein, 11 aa, Artificial Sequence
  Description of Artificial Sequence Synthetic peptide
XYGSYVSPLDY

SEQ ID NO: 29 - protein, 11 aa, Artificial Sequence
  Description of Artificial Sequence Synthetic peptide
TYGSYVSPLDY

SEQ ID NO: 30 - protein, 11 aa, Artificial Sequence
  Description of Artificial Sequence Synthetic peptide
SYGSYVSPLDY

SEQ ID NO: 31 - protein, 113 aa, Artificial Sequence
  Description of Artificial Sequence Synthetic polypeptide
LVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 32 - protein, 108 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 33 - protein, 5 aa, Artificial Sequence
  Description of Artificial Sequence Synthetic peptide
LDAQT

SEQ ID NO: 34 - protein, 9 aa, Artificial Sequence
  Description of Artificial Sequence Synthetic peptide
LTEKRKKRS

SEQ ID NO: 35 - protein, 8 aa, Artificial Sequence
  Description of Artificial Sequence Synthetic peptide
KPDSAEPM

SEQ ID NO: 36 - protein, 8 aa, Artificial Sequence
  Description of Artificial Sequence Synthetic peptide
NVRCLQHF

SEQ ID NO: 37 - protein, 123 aa, Artificial Sequence [sequence omitted]

SEQ ID NO: 38 - protein, 108 aa, Artificial Sequence; identical to SEQ ID NO: 32
  Description of Artificial Sequence Synthetic polypeptide

(19) United States
(12) Patent Application Publication
(10) Pub. No.: US 2011/0287003 A1
(43) Pub. Date: Nov. 24, 2011

(54) TREATMENT METHODS

(75) Inventors: Premal H. Patel, Redwood City, CA (US); Amy C. Peterson, San Francisco, CA (US)

(73) Assignee: Genentech, Inc., South San Francisco, CA (US)

(21) Appl. No.: 13/108,899

(22) Filed: May 16, 2011

Related U.S. Application Data
(60) Provisional application No. 61/346,424, filed on May 19, 2010.
(60) Provisional application No. 61/345,044, filed on May 14, 2010.

(51) Int. Cl. A61K 39/395; A61P 35/00

(52) U.S. Cl. 424/133.1; 424/174.1; 424/158.1

(57) ABSTRACT

The present invention relates generally to the fields of molecular biology and growth factor regulation. More specifically, the invention relates to therapies for the treatment of pathological conditions, such as cancer.

### CROSS-REFERENCE TO RELATED APPLICATIONS

This application claims priority to U.S. patent application Nos. 61/345,044, filed May 14, 2010 and 61/346,424, filed on May 19, 2010, the contents of which are incorporated herein by reference.

### SEQUENCE LISTING

This application contains a Sequence Listing which has been submitted via EFS-Web and is hereby incorporated by reference in its entirety. Said ASCII copy, created on May 10, 2011, is named P4451R1U and is 26,308 bytes in size.

### TECHNICAL FIELD

The present invention relates generally to the fields of molecular biology and growth factor regulation. More specifically, the invention relates to combination therapies for the treatment of pathological conditions, such as cancer.

### BACKGROUND

Cancer remains to be one of the most deadly threats to human health. In the U.S., cancer affects nearly 1.3 million new patients each year, and is the second leading cause of death after heart disease, accounting for approximately 1 in 4 deaths. Breast cancer is the second most common form of cancer and the second leading cancer killer among American women. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Breast cancer is a disease that kills many women each year in the United States. According to the American Cancer Society, approximately 40,000 will die from the disease in 2008. Over 180,000 new cases of breast cancer are diagnosed annually, and it is estimated that one in eight women will develop breast cancer. These numbers indicate that breast cancer is one of the most dangerous diseases facing women today. Metastatic breast cancer is generally incurable with only a few patients achieving long-term survival after standard chemotherapy. Greenberg et al., J. Clin. Oncol. 14:2197-2205 (1996).

Since cancer is still one of the most deadly threats, additional cancer treatments for patients are needed. The invention addresses these and other needs, as will be apparent upon review of the following disclosure.

All references cited herein, including patent applications and publications, are incorporated by reference in their entirety.

### SUMMARY OF THE INVENTION

In one aspect, the invention provides methods for the treatment of breast cancer, comprising administering to an estrogen receptor (ER)-negative, progesterone receptor (PR)-negative and HER2-negative (collectively termed triple-negative) metastatic breast cancer patient an effective amount of an anti-c-met antibody, and a taxane.

In one aspect, the invention provides methods for the treatment of breast cancer, comprising administering to an ER-negative, PR-negative and HER2-negative (collectively termed triple-negative) metastatic breast cancer patient an effective amount of an anti-c-met antibody, an anti-VEGF antibody, and a taxane.

In one aspect, the invention provides methods for the treatment of breast cancer, comprising administering to an ER-negative, PR-negative, and HER2-negative (ER-, PR-, and HER2-; or triple-negative) metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle, for example, to increase survival of the patient, to decrease the patient's risk of cancer recurrence and/or to increase the patient's likelihood of survival.

In yet another aspect, the invention provides methods of promoting an anti-c-met antibody (e.g., a monovalent anti-c-met antibody, e.g., MetMAb) for the treatment of a metastatic triple negative breast cancer patient, in combination with a taxane, for example, to increase survival of the patient, to decrease the patient's risk of cancer recurrence and/or to increase the patient's likelihood of survival. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle and paclitaxel administered at a dose of 90 mg/m² by IV infusion on Day 1, Day 8, and Day 15 of the 28-day cycle. Promotion may be conducted by any means available. In some embodiments, the promotion is by a package insert accompanying a commercial formulation of the anti-c-met antibody. The promotion may also be by a package insert accompanying a commercial formulation of the taxane. Promotion may be by written or oral communication to a physician or health care provider. In some embodiments, the promotion is by a package insert where the package insert provides instructions to receive therapy with anti-c-met antibody, and/or taxane. In some embodiments, the promotion is followed by the treatment of the patient with the anti-c-met antibody with or without the taxane. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

In a still further aspect, the invention provides methods for promoting a taxane for the treatment of a metastatic triple negative breast cancer patient, in combination with anti-c-met antibody, wherein the taxane may, for example, be paclitaxel, for example, to increase survival of the patient, to decrease the patient's risk of cancer recurrence and/or to increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

In some aspects, the invention features methods of instructing a patient with triple-negative metastatic breast cancer by providing instructions to receive treatment with an anti-c-met antibody, and a taxane, for example, to increase survival of the patient, to decrease the patient's risk of cancer recurrence and/or to increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

The invention provides business methods, comprising marketing an anti-c-met antibody for treatment of triple-negative metastatic breast cancer in a human patient, for example, to increase survival, decrease the patient's likelihood of cancer recurrence, and/or increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments the method further comprises marketing a taxane for treatment of triple-negative metastatic breast cancer in a human patient. In some embodiments the marketing is followed by the treatment of the patient with the anti-c-met antibody with or without the taxane.

Also provided is business methods, comprising marketing an anti-c-met antibody, and a taxane for treatment of triple-negative metastatic breast cancer in a human patient, for example, to increase survival, decrease the patient's likelihood of cancer recurrence, and/or increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments the marketing is followed by the treatment of the patient with the anti-c-met antibody with or without the taxane.

In another aspect, the invention provides articles of manufacture comprising an anti-c-met antibody (e.g., a monovalent anti-c-met antibody, e.g., MetMAb), and/or an anti-VEGF antibody, and/or a taxane, and a package insert or label with directions to treat a triple-negative metastatic breast cancer patient. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments, the treatment further comprises administering anti-VEGF antibody (e.g., bevacizumab) administered at a dose of 10 mg/kg on Day 1 and Day 15 of the 28-day cycle.

In another aspect, the invention provides a method of manufacturing an article of manufacture, wherein the article of manufacture comprises anti-c-met antibody (e.g., a monovalent anti-c-met antibody, e.g., MetMAb), and/or anti-VEGF antibody, and/or a taxane, and a package insert or label with directions to treat a triple-negative metastatic breast cancer patient. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments, the treatment further comprises administering anti-VEGF antibody (e.g., bevacizumab) administered at a dose of 10 mg/kg on Day 1 and Day 15 of the 28-day cycle.

In one aspect, the invention provides methods for the treatment of breast cancer, comprising administering to an ER-negative, PR-negative, and HER2-negative (ER-, PR-, and HER2-; or triple-negative) metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle, for example, to increase survival, decrease the patient's likelihood of cancer recurrence, and/or increase the patient's likelihood of survival. In yet another aspect, the invention provides methods comprising administration of anti-VEGF antibody. Thus, in some aspects, the invention provides methods of promoting an anti-c-met antibody (e.g., a monovalent anti-c-met antibody, e.g., MetMAb) for the treatment of a metastatic triple negative breast cancer patient, in combination with anti-VEGF antibody (e.g., bevacizumab) and a taxane, for example, to increase survival of the patient, to decrease the patient's risk of cancer recurrence and/or to increase the patient's likelihood of survival. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle. Promotion may be conducted by any means available. In some embodiments, the promotion is by a package insert accompanying a commercial formulation of the anti-c-met antibody. The promotion may also be by a package insert accompanying a commercial formulation of the anti-VEGF antibody. The promotion may also be by a package insert accompanying a commercial formulation of the taxane. Promotion may be by written or oral communication to a physician or health care provider. In some embodiments, the promotion is by a package insert where the package insert provides instructions to receive therapy with anti-c-met antibody, anti-VEGF antibody and/or taxane. In some embodiments, the promotion is followed by the treatment of the patient with the anti-c-met antibody with or without the taxane or anti-VEGF antibody. In a further aspect, the invention provides methods of promoting an anti-VEGF antibody (e.g., bevacizumab) for the treatment of a metastatic triple negative breast cancer patient, in combination with anti-c-met antibody (e.g., MetMAb) and a taxane, such as paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle.

In a still further aspect, the invention provides methods for promoting a taxane for the treatment of a metastatic triple negative breast cancer patient, in combination with anti-c-met antibody and anti-VEGF antibody, wherein the taxane may, for example, be paclitaxel, for example, to increase survival of the patient, to decrease the patient's risk of cancer recurrence and/or to increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle.

In some aspects, the invention features a method of instructing a patient with triple-negative metastatic breast cancer by providing instructions to receive treatment with an anti-c-met antibody, anti-VEGF antibody and a taxane, for example, to increase survival of the patient, to decrease the patient's risk of cancer recurrence and/or to increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle.

The invention provides a business method, comprising marketing an anti-c-met antibody for treatment of triple-negative metastatic breast cancer in a human patient, for example, to increase survival, decrease the patient's likelihood of cancer recurrence, and/or increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle. In some embodiments the method further comprises marketing an anti-VEGF antibody and a taxane for treatment of triple-negative metastatic breast cancer in a human patient. In some embodiments the marketing is followed by the treatment of the patient with the anti-c-met antibody with or without the taxane and/or anti-VEGF antibody. In some embodiments, the marketing is followed by the treatment of the patient with the anti-VEGF antibody with or without the taxane or anti-c-met antibody. Also provided is a business method, comprising marketing an anti-c-met antibody, an anti-VEGF antibody, and a taxane for treatment of triple-negative metastatic breast cancer in a human patient, for example, to increase survival, decrease the patient's likelihood of cancer recurrence, and/or increase the patient's likelihood of survival. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle. In some embodiments the marketing is followed by the treatment of the patient with the anti-c-met antibody with or without the taxane or anti-VEGF antibody. In some embodiments, the marketing is followed by the treatment of the patient with the anti-VEGF antibody with or without the taxane or anti-c-met antibody.

In another aspect, the invention provides articles of manufacture comprising an anti-c-met antibody (e.g., a monovalent anti-c-met antibody, e.g., MetMAb), and/or an anti-VEGF antibody, and/or a taxane, and a package insert or label with directions to treat a triple-negative metastatic breast cancer patient. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments, the treatment further comprises administering anti-VEGF antibody (e.g., bevacizumab) administered at a dose of 10 mg/kg on Day 1 and Day 15 of the 28-day cycle.

In another aspect, the invention provides a method of manufacturing an article of manufacture, wherein the article of manufacture comprises anti-c-met antibody (e.g., a monovalent anti-c-met antibody, e.g., MetMAb), and/or anti-VEGF antibody, and/or a taxane, and a package insert or label with directions to treat a triple-negative metastatic breast cancer patient. In some embodiments, the taxane is paclitaxel. In some embodiments, the treatment comprises administering to a triple-negative metastatic breast cancer patient an anti-c-met antibody (e.g., MetMAb) administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle. In some embodiments, the treatment further comprises administering anti-VEGF antibody (e.g., bevacizumab) administered at a dose of 10 mg/kg on Day 1 and Day 15 of the 28-day cycle.

In some embodiments, the triple-negative metastatic breast cancer patient did not receive prior treatment for triple-negative metastatic breast cancer (e.g., prior anti-cancer drug therapy). In some embodiments, the triple-negative metastatic breast cancer patient received prior treatment for triple negative metastatic breast cancer. In some embodiments, the patient has triple-negative metastatic or locally recurrent breast cancer. In some embodiments, the triple-negative metastatic or locally recurrent breast cancer patient did not receive prior treatment for triple-negative metastatic or locally recurrent breast cancer (e.g., prior anti-cancer drug therapy). In some embodiments, the triple-negative metastatic or locally recurrent breast cancer patient received prior treatment for triple negative metastatic or locally recurrent breast cancer.

In a further embodiment, the anti-c-met antibody, and taxane are administered concurrently. In a still further embodiment, the anti-c-met antibody, and the taxane are administered consecutively, in any order. In another embodiment, administration of the anti-c-met antibody precedes administration of the taxane. In a further embodiment, administration of the anti-c-met antibody (e.g., MetMAb), and taxane (e.g., paclitaxel) results in a synergistic effect. In a still further embodiment, administration of the anti-c-met antibody (e.g., MetMAb), and taxane (e.g., paclitaxel) extends survival of the human patient relative to treatment in the absence of anti-c-met antibody. In a particular embodiment, progression free survival (PFS) and/or overall survival (OS) is extended. In some embodiments, the treatment extends PFS or OS at least about 5%, at least about 10%, at least about 15%, at least about 20% or more than PFS or OS achieved by administering taxane to the patient.

In a further embodiment, the anti-c-met antibody, anti-VEGF antibody and taxane are administered concurrently. In a still further embodiment, the anti-c-met antibody, anti-VEGF antibody and the taxane are administered consecutively, in any order. In another embodiment, administration of the anti-c-met antibody precedes administration of the anti-VEGF antibody and the taxane. In a further embodiment, administration of the anti-c-met antibody (e.g., MetMAb), anti-VEGF antibody (e.g., bevacizumab) and taxane (e.g., paclitaxel) results in a synergistic effect. In a still further embodiment, administration of the anti-c-met antibody (e.g., MetMAb), anti-VEGF antibody (e.g., bevacizumab) and taxane (e.g., paclitaxel) extends survival of the human patient relative to treatment in the absence of anti-c-met antibody. In a particular embodiment, progression free survival (PFS) and/or overall survival (OS) is extended. In some embodiments, the treatment extends PFS or OS at least about 5%, at least about 10%, at least about 15%, at least about 20% or more than PFS or OS achieved by administering anti-VEGF antibody and taxane to the patient.

Although the methods of the present invention may be performed in the absence of any other means of cancer therapy, e.g. in the absence of a further therapeutic agent, including chemotherapeutic agents, the methods may optionally comprise the administration of a further therapeutic agent selected from the group consisting of chemotherapeutic agent, a different anti-c-met antibody, a different anti-VEGF antibody, antibody directed against a tumor associated antigen, anti-hormonal compound, cardioprotectant, cytokine, anti-angiogenic agent, tyrosine kinase inhibitor, COX inhibitor, non-steroidal anti-inflammatory drug, farnesyl transferase inhibitor, antibody that binds oncofetal protein CA 125, Raf or ras inhibitor, liposomal doxorubicin, topotecan, a different taxane, a medicament that treats nausea, a medicament that prevents or treats skin rash or standard acne therapy, a medicament that treats or prevents diarrhea, a body temperature-reducing medicament, and a hematopoietic growth factor.

In a further aspect of the invention, the taxane according to any of the embodiments herein is, for example, TAXOL® paclitaxel (Bristol-Myers Squibb Oncology, Princeton, N.J.), TAXOTERE® docetaxel (Rhône-Poulenc Rorer, Antony, France), or ABRAXANE™ Cremophor-free, albumin-engineered nanoparticle formulation of paclitaxel (American Pharmaceutical Partners, Schaumberg, Ill.). In some embodiments, the taxane is paclitaxel administered at a dose of 90 mg/m² by IV infusion on Day 1, Day 8, and Day 15 of each 28-day cycle. Two or more taxanes can be used in a cocktail to be administered in combination with administration with the anti-c-met antibody and anti-VEGF antibody.

In a further aspect of the invention, an antibody according to any of the embodiments herein is a monoclonal antibody, including a chimeric, humanized or human antibody. In one embodiment, an antibody is an antibody fragment, e.g., a Fv, Fab, Fab′, scFv, diabody, a one-armed antibody, or F(ab′)₂ fragment. In another embodiment, the antibody is a full length antibody, e.g., an intact IgG antibody or other antibody class or isotype as defined herein. In another embodiment, the antibody is a naked antibody. In still another embodiment, the antibody is conjugated to a drug.

In another aspect of the invention, the anti-c-met antibody is a monovalent, one-armed antibody. The present application discloses administration in humans of a monovalent one-armed antibody comprising a Fc region that increases stability of said antibody fragment compared to a Fab molecule comprising said antigen binding arm. See, e.g., WO2005/063816. A full length antibody may in some cases exhibit agonistic effects (which may be undesirable) upon binding to a target antigen even though it is an antagonistic antibody as a Fab fragment. See, e.g., U.S. Pat. No. 6,468,529. This phenomenon is unfortunate where the antagonistic effect is the desired therapeutic function. In these cases, the monovalent trait of a one-armed antibody (i.e., an antibody comprising a single antigen binding arm) results in and/or ensures an antagonistic function upon binding of the antibody to a target molecule, suitable for treatment of pathological conditions requiring an antagonistic function and where bivalency of an antibody results in an undesirable agonistic effect. Furthermore, a one-armed antibody comprising the Fc region as described herein is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies. Accordingly, in some embodiments, the anti-c-met antibody is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm.

In some embodiments, the anti-c-met antibody is an anti-c-met antibody or antibody fragment thereof, wherein the antibody comprises (a) a first polypeptide comprising a heavy chain variable domain comprising the sequence: EVQLVESGGGLVQPGGSLRLSCAASGYTFTSYWLHWVRQAPGKGLEWVGMIDPSN SDTRFNPNFKDRFTISADTSKNTAYLQMNSLRAEDTAVYYCATYRSYVTPLDYWGQ GTLVTVSS (SEQ ID NO:1), CH1 sequence, and a first Fc polypeptide; (b) a second polypeptide comprising a light chain variable domain comprising the sequence: DIQMTQSPSSLSASVGDRVTITCKSSQSLLYTSSQKNYLAWYQQKPGKAPKLLIYWA STR ESGVPSRFSGSGSGTDFTLTISSLQPEDFATYYCQQYYAYPWTFGQGTKVEIKR (SEQ ID NO:2), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm. In some embodiments, the first polypeptide comprises the Fc sequence depicted in FIG. 1 (SEQ ID NO: 3) and the second polypeptide comprises the Fc sequence depicted in FIG. 2 (SEQ ID NO: 4). In some embodiments, the first polypeptide comprises the Fc sequence depicted in FIG. 2 (SEQ ID NO: 4) and the second polypeptide comprises the Fc sequence depicted in FIG. 1 (SEQ ID NO: 3).

In some embodiments, the anti-c-met antibody is an anti-c-met antibody or antibody fragment thereof, wherein the antibody comprises (a) a first polypeptide comprising a heavy chain variable domain, said polypeptide comprising the sequence: EVQLVESGGGLVQPGGSLRLSCAASGYTFTSYWLHWVRQAPGKGLEWVGMIDPSN SDTRFNPNFKDRFTISADTSKNTAYLQMNSLRAEDTAVYYCATYRSYVTPLDYWGQ GTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHT CPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISK AKGQPREPQVYTLPPSREEMTKNQVSLSCAVKGFYPSDIAVEWESNGQPENNYKTTP PVLDSDGSFFLVSKLTVDKSRWQQGNVFSCSVMHEALHNHYTQKSLSLSPGK (SEQ ID NO: 21); (b) a second polypeptide comprising a light chain variable domain, the polypeptide comprising the sequence DIQMTQSPSSLSASVGDRVTITCKSSQSLLYTSSQKNYLAWYQQKPGKAPKLLIYWA STRESGVPSRFSGSGSGTDFTLTISSLQPEDFATYYCQQYYAYPWTFGQGTKVEIKRT VAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 22); and a third polypeptide comprising a Fc sequence, the polypeptide comprising the sequence DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSREEMTKNQVSLWCLVKGFYPSDIAVEWESNGQPENN YKTTPPVLDSDGSFFLYSKLTVDKSRWQQGNVFSCSVMHEALHNHYTQKSLSLSPG K (SEQ ID NO: 4), wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

In one embodiment, the anti-c-met antibody comprises a heavy chain variable domain comprising one or more of CDR1-HC, CDR2-HC and CDR3-HC sequence depicted in FIG. 1 (SEQ ID NOs: 5, 6, 7). In some embodiments, the antibody comprises a light chain variable domain comprising one or more of CDR1-LC, CDR2-LC and CDR3-LC sequence depicted in FIG. 1 (SEQ ID NOs: 8, 9, 10). In some embodiments, the heavy chain variable domain comprises FR1-HC, FR2-HC, FR3-HC and FR4-HC sequence depicted in FIG. 1 (SEQ ID NOs: 11, 12, 13, 14). In some embodiments, the light chain variable domain comprises FR1-LC, FR2-LC, FR3-LC and FR4-LC sequence depicted in FIG. 1 (SEQ ID NOs: 15, 16, 17, 18).

In some embodiments, the anti-c-met antibody is onartuzumab (interchangeably termed MetMAb).

Other anti-c-met antibodies suitable for use in the methods of the invention are described herein and known in the art. Anti-hepatocyte growth factor (HGF) antibodies are also suitable for use in the methods of the invention involving anti-c-met antibodies (either in combination with anti-c-met antibody or substituting for anti-c-met antibody). As is known in the art, HGF is a ligand for c-met receptor.

In some embodiments, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816. For example, a hole mutation can be one or more of T366A, L368A and/or Y407V in an Fc polypeptide, and a knob mutation can be T366W.

In another aspect, the anti-VEGF antibody according to any of the embodiments herein may be substituted with a VEGF specific antagonist, e.g., a VEGF receptor molecule or chimeric VEGF receptor molecule as described below. In certain embodiments of the methods of the invention, the anti-VEGF antibody is bevacizumab. Exemplary antibodies useful in the methods of the invention include bevacizumab (AVASTIN®), a G6 antibody, a B20 antibody, and fragments thereof. In certain embodiments, the anti-VEGF antibody has a heavy chain variable region comprising the following amino acid sequence: EVQLVESGGG LVQPGGSLRL SCAASGYTFT NYGMNWVRQA PGKGLEWVGW INTYTGEPTY AADFKRRFTF SLDTSKSTAY LQMNSLRAED TAVYYCAKYP HYYGSSHWYF DVWGQGTLVT VSS (SEQ ID NO: 31) and a light chain variable region comprising the following amino acid sequence: DIQMTQSPSS LSASVGDRVT ITCSASQDIS NYLNWYQQKP GKAPKVLIYFTSSLHSGVPS RFSGSGSGTD FTLTISSLQP EDFATYYCQQ YSTVPWTFGQGTKVEIKR (SEQ ID NO: 32).

In another aspect of the invention, in some embodiments of the methods provided herein, the patient's cancer expresses c-met. In some embodiments, serum from a patient expresses high levels of IL8 (displays high levels of IL8 expression, such as IL8 protein expression). In some embodiments, serum from a patient expresses greater than about 150 pg/ml of IL8, or in some embodiments, greater than about 50 pg/ml IL8. In some embodiments, serum from a patient expresses greater than about 10 pg/ml, 20 pg/ml, 30 pg/ml or more of IL8. Methods for determining IL8 serum concentration are known in the art. In some embodiments, serum from a patient expresses high levels of HGF (displays high level of HGF expression, such as HGF protein expression). In some embodiments, serum from a patient expresses greater than about 5,000, 10,000, or 50,000 pg/ml of HGF.

### BRIEF DESCRIPTION OF THE DRAWINGS

FIG. 1: depicts amino acid sequences of the framework (FR), CDR, first constant domain (CL or CH1) and Fc region (Fc) of MetMAb (onartuzumab, or OA5D5v2). The Fc sequence depicted comprises “hole” (cavity) mutations T366S, L368A and Y407V, as described in WO 2005/063816.

FIG. 2: depicts sequence of an Fc polypeptide comprising “knob” (protuberance) mutation T366W, as described in WO 2005/063816. In one embodiment, an Fc polypeptide comprising this sequence forms a complex with an Fc polypeptide comprising the Fc sequence of FIG. 1 to generate an Fc region.

FIG. 3: depicts patient diagnosis, treatment cohort and administered cycles. BEV=bevacizumab; CR=complete response; *=dose-limiting toxicity.

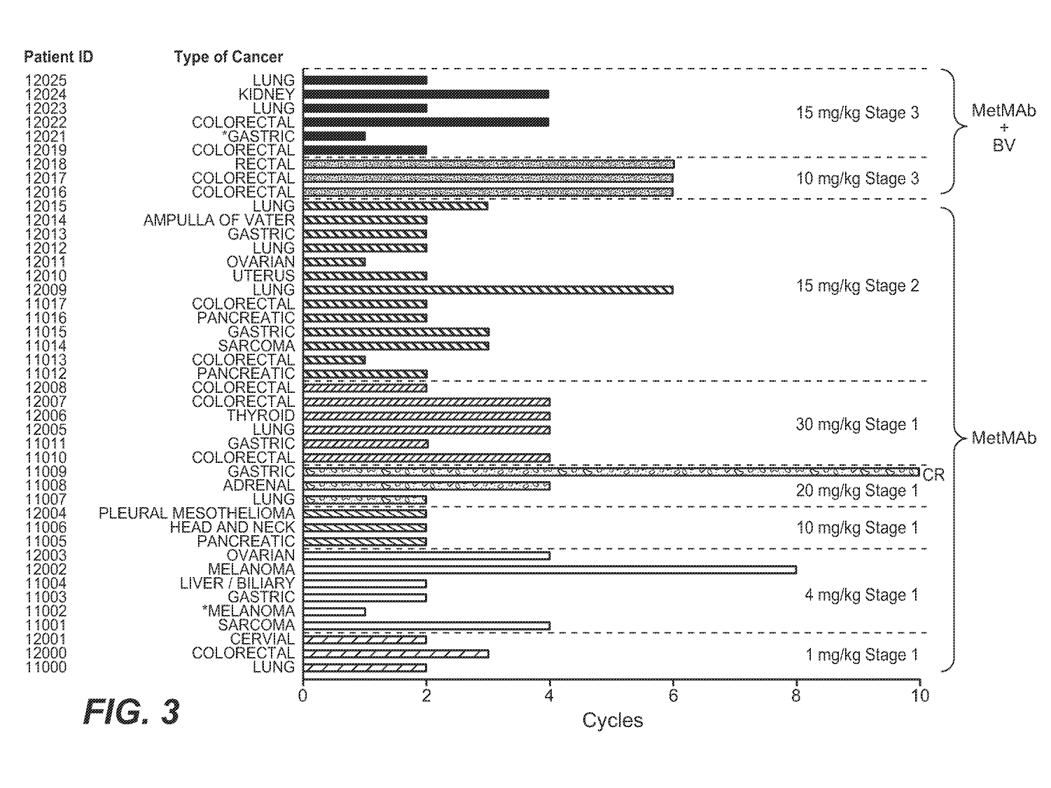

FIG. 4: depicts change of tumor burden from baseline with best response, all patients. SLD=sum of longest diameter; *=no data due to dose-limiting toxicity; **=not done.

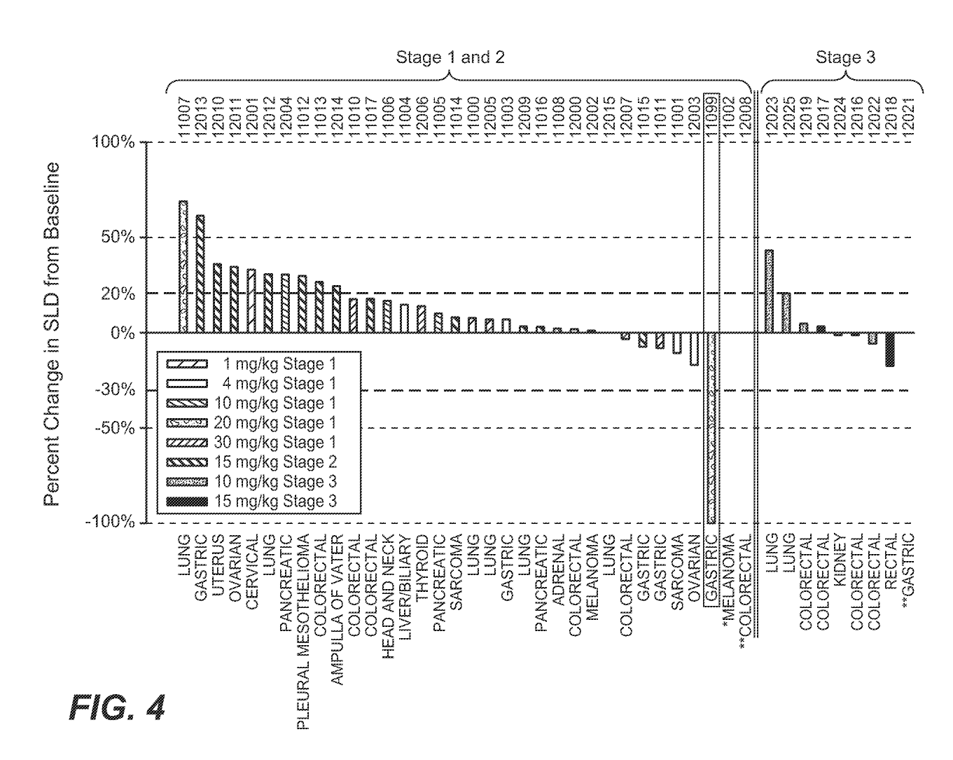

### DETAILED DESCRIPTION

### I. Definitions

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur. The term “wild type HGF” generally refers to a polypeptide comprising the amino acid sequence of a naturally occurring HGF protein. The term “wild type HGF sequence” generally refers to an amino acid sequence found in a naturally occurring HGF. C-met is a known receptor for HGF through which HGF intracellular signaling is biologically effectuated.

The term “estrogen receptor” or “ER” as used herein, refers, unless indicated otherwise, to any native or variant (whether native or synthetic) ER polypeptide. The term “wild type ER” generally refers to a polypeptide comprising the amino acid sequence of a naturally occurring ER protein. The term “wild type ER sequence” generally refers to an amino acid sequence found in a naturally occurring ER.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

The term “progesterone receptor” or “PR”, as used herein, refers, unless indicated otherwise, to any native or variant (whether native or synthetic) PR polypeptide. The term “wild type PR” generally refers to a polypeptide comprising the amino acid sequence of a naturally occurring PR protein. The term “wild type PR sequence” generally refers to an amino acid sequence found in a naturally occurring PR.

A “native sequence” polypeptide comprises a polypeptide having the same amino acid sequence as a polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

An “anti-c-met antibody” is an antibody that binds to c-met with sufficient affinity and specificity. The antibody selected will normally have a sufficiently strong binding affinity for c-met, for example, the antibody may bind human c-met with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-c-met antibody can be used as a therapeutic agent in targeting and interfering with diseases or conditions wherein c-met activity is involved. Also, the antibody may be subjected to other biological activity assays, e.g., in order to evaluate its effectiveness as a therapeutic. Such assays are known in the art and depend on the target antigen and intended use for the antibody.

A “tyrosine kinase inhibitor” is a molecule which inhibits to some extent tyrosine kinase activity of a tyrosine kinase such as a c-met receptor.

Protein “expression” refers to conversion of the information encoded in a gene into messenger RNA (mRNA) and then to the protein. Herein, a sample or cell that “expresses” a protein of interest (such as a c-met protein) is one in which mRNA encoding the protein, or the protein, including fragments thereof, is determined to be present in the sample or cell. The term “interleukin 8” or “IL8” or “IL-8”, as used herein, refers, unless indicated otherwise, to any native or variant (whether native or synthetic) IL8 polypeptide that is capable of activating the IL8 signaling pathway under conditions that permit such process to occur. The term “wild type IL8” generally refers to a polypeptide comprising the amino acid sequence of a naturally occurring IL8 protein. The term “wild type IL8 sequence” generally refers to an amino acid sequence found in a naturally occurring IL8.

The term “VEGF” or “VEGF-A” is used to refer to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof. VEGF-A is part of a gene family including VEGF-B, VEGF-C, VEGF-D, VEGF-E, VEGF-F, and PlGF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. Additionally, neuropilin-1 has been identified as a receptor for heparin-binding VEGF-A isoforms, and may play a role in vascular development. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

An “angiogenesis inhibitor” or “anti-angiogenesis agent” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or to the VEGF-A receptor (e.g., KDR receptor or Flt-1 receptor), anti-PDGFR inhibitors such as GLEEVEC® (Imatinib Mesylate). Anti-angiogenesis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials.

A “VEGF antagonist” refers to a molecule (peptidyl or non-peptidyl) capable of neutralizing, blocking, inhibiting, abrogating, reducing, or interfering with VEGF activities including its binding to one or more VEGF receptors. In certain embodiments, the VEGF antagonist reduces or inhibits, by at least 10%, 20%, 30%, 40%, 50%, 60%, 70%, 80%, 90% or more, the expression level or biological activity of VEGF. In one embodiment, the VEGF inhibited by the VEGF antagonist is VEGF (8-109), VEGF (1-109), or VEGF₁₆₅. VEGF antagonists useful in the methods of the invention include peptidyl or non-peptidyl compounds that specifically bind VEGF, such as anti-VEGF antibodies and antigen-binding fragments thereof, polypeptides, or fragments thereof that specifically bind to VEGF, and receptor molecules and derivatives that bind specifically to VEGF thereby sequestering its binding to one or more receptors (e.g., soluble VEGF receptor proteins, or VEGF binding fragments thereof, or chimeric VEGF receptor proteins); antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers.

An “anti-VEGF antibody” is an antibody that binds to VEGF with sufficient affinity and specificity. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-VEGF antibody of the invention can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay (as described in the Examples below); tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PlGF, PDGF or bFGF.

In certain embodiments, anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. Cancer Res. 57:4593-4599 (1997). In one embodiment, the anti-VEGF antibody is “Bevacizumab (BV)”, also known as “rhuMAb VEGF” or “AVASTIN®”. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab has been approved by the FDA for use in combination with chemotherapy regimens to treat metastatic colorectal cancer (CRC) and non-small cell lung cancer (NSCLC). Hurwitz et al., N. Engl. J. Med. 350:2335-42 (2004); Sandler et al., N. Engl. J. Med. 355:2542-50 (2006). Currently, bevacizumab is being investigated in many ongoing clinical trials for treating various cancer indications. Kerbel, J. Clin. Oncol. 19:45 S-51S (2001); De Vore et al, Proc. Am. Soc. Clin. Oncol. 19:485a. (2000); Hurwitz et al., Clin. Colorectal Cancer 6:66-69 (2006); Johnson et al., Proc. Am. Soc. Clin. Oncol. 20:315a (2001); Kabbinavar et al. J. Clin. Oncol. 21:60-65 (2003); Miller et al., Breast Can. Res. Treat. 94:Suppl 1:S6 (2005).

Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, and U.S. Patent Application 60/991,302, the content of these patent applications are expressly incorporated herein by reference. For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, I83 and Q89.

A “G6 series antibody” according to this invention, is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, I83 and Q89.

A “B20 series antibody” according to this invention is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and U.S. Patent Application 60/991,302, the content of these patent applications are expressly incorporated herein by reference. In one embodiment, the B20 series antibody binds to a functional epitope on human VEGF comprising residues F17, M18, D19, Y21, Y25, Q89, I91, K101, E103, and C104.

A “functional epitope” according to this invention refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC50 mutant VEGF/IC50 wild-type VEGF) of the antibody will be greater than 5 (see Example 2 of WO2005/012359). In one embodiment, the relative affinity ratio is determined by a solution binding phage displaying ELISA. Briefly, 96-well Maxisorp immunoplates (NUNC) are coated overnight at 4° C. with an Fab form of the antibody to be tested at a concentration of 2 ug/ml in PBS, and blocked with PBS, 0.5% BSA, and 0.05% Tween20 (PBT) for 2 h at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′,5,5′-tetramethylbenzidine (TMB, Kirkegaard & Perry Labs, Gaithersburg, Md.) substrate for approximately 5 min, quenched with 1.0 M H3PO4, and read spectrophotometrically at 450 nm. The ratio of IC50 values (IC50, ala/IC50, wt) represents the fold of reduction in binding affinity (the relative binding affinity).

An “immunoconjugate” (interchangeably referred to as “antibody-drug conjugate,” or “ADC”) means an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. In one embodiment, an antibody of the invention is a one-armed antibody as described in WO2005/063816. In one embodiment, the one-armed antibody comprises Fc mutations constituting “knobs” and “holes” as described in WO2005/063816. For example, a hole mutation can be one or more of T366A, L368A and/or Y407V in an Fc polypeptide, and a knob mutation can be T366W.

A “blocking” antibody or an antibody “antagonist” is one which inhibits or reduces biological activity of the antigen it binds. In some embodiments, blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

The phrase “antigen binding arm”, as used herein, refers to a component part of an antibody fragment of the invention that has an ability to specifically bind a target molecule of interest. Generally and preferably, the antigen binding arm is a complex of immunoglobulin polypeptide sequences, e.g., CDR and/or variable domain sequences of an immunoglobulin light and heavy chain.

The phrase “N-terminally truncated heavy chain”, as used herein, refers to a polypeptide comprising parts but not all of a full length immunoglobulin heavy chain, wherein the missing parts are those normally located on the N terminal region of the heavy chain. Missing parts may include, but are not limited to, the variable domain, CH1, and part or all of a hinge sequence. Generally, if the wild type hinge sequence is not present, the remaining constant domain(s) in the N-terminally truncated heavy chain would comprise a component that is capable of linkage to another Fc sequence (i.e., the “first” Fc polypeptide as described herein). For example, said component can be a modified residue or an added cysteine residue capable of forming a disulfide linkage.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The C-terminal lysine (residue 447 according to the EU numbering system) of the Fc sequence may be removed, for example, during purification of the antibody or by recombinant engineering of the nucleic acid encoding the antibody. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

A “naked antibody” is an antibody that is not conjugated to a heterologous molecule, such as a cytotoxic moiety or radiolabel.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K_{d}) value of no more than about 1×10⁻⁷ M, preferably no more than about 1×10⁻⁸ M and most preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above. In one embodiment, the species-dependent antibody is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e. same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

A “chimeric VEGF receptor protein” is a VEGF receptor molecule having amino acid sequences derived from at least two different proteins, at least one of which is as VEGF receptor protein. In certain embodiments, the chimeric VEGF receptor protein is capable of binding to and inhibiting the biological activity of VEGF.

An “isolated” polypeptide or “isolated” antibody is one that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or nonproteinaceous solutes. In preferred embodiments, the polypeptide or antibody will be purified (1) to greater than 95% by weight of polypeptide or antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide or antibody includes the polypeptide or antibody in situ within recombinant cells since at least one component of the polypeptide's natural environment will not be present. Ordinarily, however, isolated polypeptide or antibody will be prepared by at least one purification step.

The term “biomarker” or “marker” as used herein refers generally to a molecule, including a gene, mRNA, protein, carbohydrate structure, or glycolipid, the expression of which in or on a tissue or cell or secreted can be detected by known methods (or methods disclosed herein) and is predictive or can be used to predict (or aid prediction) for a cell, tissue, or patient's responsiveness to treatment regimes. By “patient sample” is meant a collection of similar cells obtained from a cancer patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsies, circulating tumor cells, serum or plasma, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In one embodiment the sample comprises 3N MBC tumor sample.

An “effective response” of a patient or a patient's “responsiveness” to treatment with a medicament and similar wording refers to the clinical or therapeutic benefit imparted to a patient at risk for, or suffering from, cancer (e.g., 3N MBC) upon administration of the cancer medicament. Such benefit includes any one or more of: extending survival (including overall survival and progression free survival); resulting in an objective response (including a complete response or a partial response); or improving signs or symptoms of cancer, etc. In one embodiment, a biomarker (e.g., c-met expression, for example, as determined using IHC) is used to identify the patient who is expected to have greater progression free survival (PFS) when treated with a medicament (e.g., anti-c-met antibody), relative to a patient who does not express the biomarker at the same level. In one embodiment, the biomarker is used to identify the patient who is expected to have greater overall survival (OS) when treated with a medicament, relative to a patient who does not express the biomarker at the same level. The incidence of biomarker(s) herein effectively predicts, or predicts with high sensitivity, such effective response.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer. In some embodiments, the cancer is triple-negative metastatic breast cancer, including any histologically confirmed triple-negative (ER-, PR-, HER2-) adenocarcinoma of the breast with locally recurrent or metastatic disease, e.g., where the locally recurrent disease is not amenable to resection with curative intent.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Herein “time to disease progression” or “TTP” refer to the time, generally measured in weeks or months, from the time of initial treatment (e.g. with a anti-c-met antibody, e.g. MetMAb), until the cancer progresses or worsens. Such progression can be evaluated by the skilled clinician. In the case of triple-negative metastatic breast cancer, for instance, progression can be evaluated by RECIST.

By “extending TTP” is meant increasing the time to disease progression in a treated patient relative to an untreated patient (i.e. relative to a patient not treated with a anti-c-met antibody, such as MetMAb), and/or relative to a patient treated with an approved anti-tumor agent.

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with anti-c-met antibody, such as MetMAb), and/or relative to a patient treated with an approved anti-tumor agent.

An “objective response” refers to a measurable response, including complete response (CR) or partial response.

By “complete response” or “CR” is intended the disappearance of all signs of cancer in response to treatment. This does not always mean the cancer has been cured.

“Partial response” refers to a decrease in the size of one or more tumors or lesions, or in the extent of cancer in the body, in response to treatment.

By “primary tumor” or “primary cancer” is meant the original cancer and not a metastatic lesion located in another tissue, organ, or location in the subject's body.

By “subject” is meant a mammal, including, but not limited to, a human or non-human mammal, such as a bovine, equine, canine, ovine, or feline. Preferably, the subject is a human. Patients are also subjects herein.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON•toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

By “extending survival” or “increasing the likelihood of survival” is meant increasing PFS and/or OS in a treated patient relative to an untreated patient (e.g., relative to a patient not treated with an anti-c-met antibody), or relative to a control treatment protocol, such as treatment only with the chemotherapeutic agent, such as those use in the care for breast cancer. Survival is monitored for at least about one month, two months, four months, six months, nine months, or at least about 1 year, or at least about 2 years, or at least about 3 years, or at least about 4 years, or at least about 5 years, or at least about 10 years, etc., following the initiation of treatment or following the initial diagnosis.

For the methods of the invention, the term “instructing” a patient means providing directions for applicable therapy, medication, treatment, treatment regimens, and the like, by any means, but preferably in writing, such as in the form of package inserts or other written promotional material.

For the methods of the invention, the term “promoting” means offering, advertising, selling, or describing a particular drug, combination of drugs, or treatment modality, by any means, including writing, such as in the form of package inserts. Promoting herein refers to promotion of therapeutic agent(s), such as an anti-c-met antibody, an anti-VEGF antibody and a taxane, or such as an anti-c-met antibody, and a taxane, for an indication, such as breast cancer treatment, where such promoting is authorized by the Food and Drug Administration (FDA) as having been demonstrated to be associated with statistically significant therapeutic efficacy and acceptable safety in a population of subjects

The term “marketing” is used herein to describe the promotion, selling or distribution of a product (e.g., drug). Marketing specifically includes packaging, advertising, and any business activity with the purpose of commercializing a product.

A “population” of subjects refers to a group of subjects with cancer, such as in a clinical trial, or as seen by oncologists following FDA approval for a particular indication, such as breast cancer therapy.

The term “intravenous infusion” refers to introduction of a drug into the vein of an animal or human patient over a period of time greater than approximately 5 minutes, preferably between approximately 30 to 90 minutes, although, according to the invention, intravenous infusion is alternatively administered for 10 hours or less.

The term “intravenous bolus” or “intravenous push” refers to drug administration into a vein of an animal or human such that the body receives the drug in approximately 15 minutes or less, preferably 5 minutes or less.

The term “subcutaneous administration” refers to introduction of a drug under the skin of an animal or human patient, preferable within a pocket between the skin and underlying tissue, by relatively slow, sustained delivery from a drug receptacle. The pocket may be created by pinching or drawing the skin up and away from underlying tissue.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is preferably less than approximately 15 minutes, more preferably less than 5 minutes, and most preferably less than 60 seconds. Administration is preferably within a pocket between the skin and underlying tissue, where the pocket is created, for example, by pinching or drawing the skin up and away from underlying tissue.

### Therapeutic Agents

The present invention features, for example, the use of anti-c-met antibodies and a taxane in combination therapy to treat a pathological condition, such as triple-negative metastatic breast cancer, in a patient. The present invention also features, for example, the use of anti-c-met antibodies, VEGF antagonists (such as anti-VEGF antibodies) and a taxane in combination therapy to treat a pathological condition, such as triple-negative metastatic breast cancer, in a patient.

### Anti-c-met Antibodies

Anti-c-met antibodies that are useful in the methods of the invention include any antibody that binds with sufficient affinity and specificity to c-met and can reduce or inhibit one or more c-met activities. Anti-c-met antibodies can be used to modulate one or more aspects of HGF/c-met-associated effects, including but not limited to c-met activation, downstream molecular signaling (e.g., mitogen activated protein kinase (MAPK) phosphorylation), cell proliferation, cell migration, cell survival, cell morphogenesis and angiogenesis. These effects can be modulated by any biologically relevant mechanism, including disruption of ligand (e.g., HGF) binding to c-met, c-met phosphorylation and/or c-met multimerization.

The antibody selected will normally have a sufficiently strong binding affinity for c-met, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein c-met/HGF activity is involved. Also, the antibody may be subjected to other biological activity assays, e.g., in order to evaluate its effectiveness as a therapeutic. Such assays are known in the art and depend on the target antigen and intended use for the antibody.

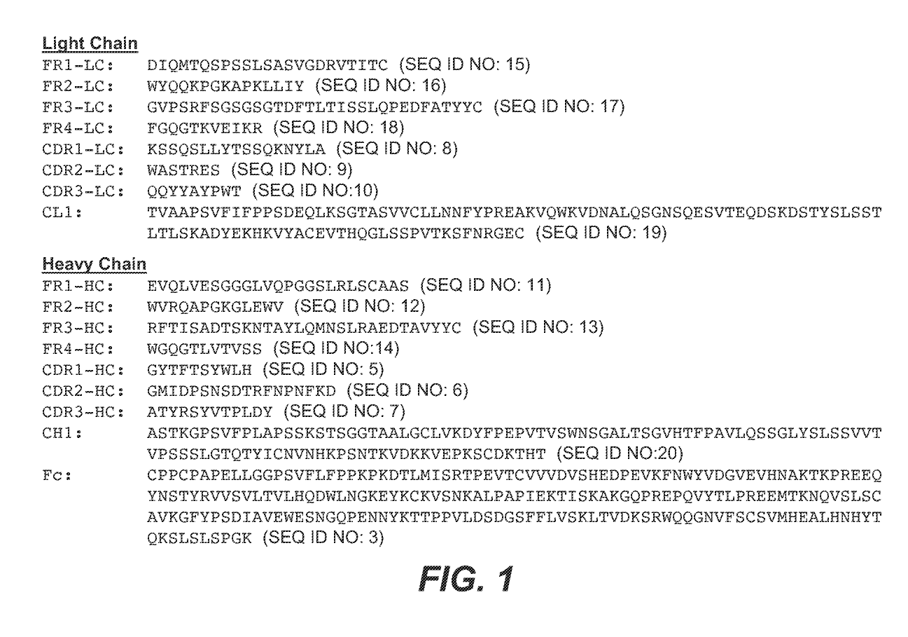

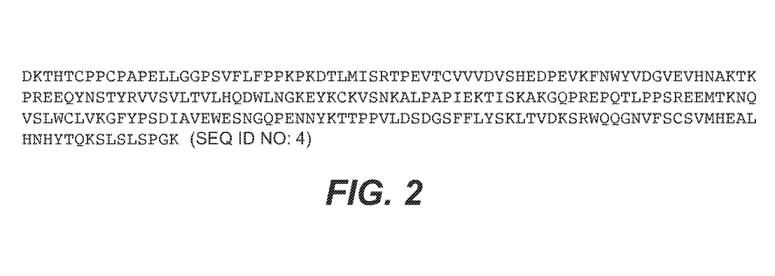

Anti-c-met antibodies (which may provided as one-armed antibodies) are known in the art. See, e.g., Martens, T, et al (2006) Clin Cancer Res 12(20 Pt 1):6144; U.S. Pat. No. 6,468,529; WO2006/015371; WO2007/063816. The present application discloses administration of onartuzumab (interchangeably termed “MetMAb”), a one-armed antibody comprising a Fc region, in humans. A sequence of MetMAb is shown in FIGS. 1 and 2. MetMAb (also termed OA5D5v2 and onartuzumab) is also described in, e.g., WO2006/015371; Jin et al, Cancer Res (2008) 68:4360. Administration of a biosimilar version of MetMAb is also contemplated by the invention. Exemplary anti-c-met antibodies are also described and exemplified herein.

Use of anti-Hepatocyte Growth Factor (HGF) antibodies is also contemplated by the invention. HGF is the ligand for the c-met receptor. Anti-HGF antibodies are known in the art. See, e.g., Kim K J, et al. Clin Cancer Res. (2006) 12(4):1292-8; WO2007/115049; WO2009/002521; WO2007/143098; WO2007/017107; WO2005/017107; L2G7; AMG-102 (rilotumumab), AV-299. Anti-HGF antibodies may be administered in addition to anti-c-met antibodies, or in substitution for anti-c-met antibodies.

In some embodiments, the invention provides for use of anti-c-met antibodies described herein or known in the art, in the one-armed format. Accordingly, in one aspect, the anti-c-met antibody is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm. For treatment of pathological conditions requiring an antagonistic function, and where bivalency of an antibody results in an undesirable agonistic effect, the monovalent trait of a one-armed antibody (i.e., an antibody comprising a single antigen binding arm) results in and/or ensures an antagonistic function upon binding of the antibody to a target molecule. Furthermore, the one-armed antibody comprising a Fc region is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies. One-armed antibodies are disclosed in, for example, WO2005/063816; Martens et al, Clin Cancer Res (2006), 12: 6144.

In some embodiments, the anti-c-met antibody is an anti-c-met antibody or antibody fragment thereof, wherein the anti-c-met antibody comprises (a) a first polypeptide comprising a heavy chain variable domain, said polypeptide comprising the sequence: EVQLVESGGGLVQPGGSLRLSCAASGYTFTSYWLHWVRQAPGKGLEWVGMIDPSN SDTRFNPNFKDRFTISADTSKNTAYLQMNSLRAEDTAVYYCATYRSYVTPLDYWGQ GTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHT CPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISK AKGQPREPQVYTLPPSREEMTKNQVSLSCAVKGFYPSDIAVEWESNGQPENNYKTTP PVLDSDGSFFLVSKLTVDKSRWQQGNVFSCSVMHEALHNHYTQKSLSLSPGK (SEQ ID NO: 21); (b) a second polypeptide comprising a light chain variable domain, the polypeptide comprising the sequence DIQMTQSPSSLSASVGDRVTITCKSSQSLLYTSSQKNYLAWYQQKPGKAPKLLIYWA STRESGVPSRFSGSGSGTDFTLTISSLQPEDFATYYCQQYYAYPWTFGQGTKVEIKRT VAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 22); and a third polypeptide comprising a Fc sequence, the polypeptide comprising the sequence DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSREEMTKNQVSLWCLVKGFYPSDIAVEWESNGQPENN YKTTPPVLDSDGSFFLYSKLTVDKSRWQQGNVFSCSVMHEALHNHYTQKSLSLSPG K (SEQ ID NO: 4), wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

In one embodiment, the anti-c-met antibody comprises a heavy chain variable domain comprising one or more of CDR1-HC, CDR2-HC and CDR3-HC sequence depicted in FIG. 1 (SEQ ID NOS 5-7). In some embodiments, the antibody comprises a light chain variable domain comprising one or more of CDR1-LC, CDR2-LC and CDR3-LC sequence depicted in FIG. 1 (SEQ ID NOS 8-10). In some embodiments, the heavy chain variable domain comprises FR1-HC, FR2-HC, FR3-HC and FR4-HC sequence depicted in FIG. 1 (SEQ ID NOS 11-14). In some embodiments, the light chain variable domain comprises FR1-LC, FR2-LC, FR3-LC and FR4-LC sequence depicted in FIG. 1 (SEQ ID NOS 15-18).

In other embodiments, the antibody comprises one or more of the CDR sequences of the monoclonal antibody produced by the hybridoma cell line deposited under American Type Culture Collection Accession Number ATCC HB-11894 (hybridoma 1A3.3.13) or HB-11895 (hybridoma 5D5.11.6).

In one aspect, the anti-c-met antibody comprises: (a) at least one, two, three, four or five hypervariable region (CDR) sequences selected from the group consisting of: (i) CDR-L1 comprising sequence A1-A17, wherein A1-A17 is KSSQSLLYTSSQKNYLA (SEQ ID NO:23) (ii) CDR-L2 comprising sequence B1-B7, wherein B1-B7 is WASTRES (SEQ ID NO:24); (iii) CDR-L3 comprising sequence C1-C9, wherein C1-C9 is QQYYAYPWT (SEQ ID NO:25); (iv) CDR-H1 comprising sequence D1-D10, wherein D1-D10 is GYTFTSYWLH (SEQ ID NO:26); (v) CDR-H2 comprising sequence E1-E18, wherein E1-E18 is GMIDPSNSDTRFNPNFKD (SEQ ID NO:27); and (vi) CDR-H3 comprising sequence F1-F11, wherein F1-F11 is XYGSYVSPLDY (SEQ ID NO:28) and X is not R; and (b) at least one variant CDR, wherein the variant CDR sequence comprises modification of at least one residue of the sequence depicted in SEQ ID NOs:23, 24, 25, 26, 27, or 28. In one embodiment, CDR-L1 of an antibody of the invention comprises the sequence of SEQ ID NO:23. In one embodiment, CDR-L2 comprises the sequence of SEQ ID NO:24. In one embodiment, CDR-L3 comprises the sequence of SEQ ID NO:25. In one embodiment, CDR-H1 comprises the sequence of SEQ ID NO:26. In one embodiment, CDR-H2 comprises the sequence of SEQ ID NO:27. In one embodiment, CDR-H3 the sequence of SEQ ID NO:28. In one embodiment, CDR-H3 comprises TYGSYVSPLDY (SEQ ID NO: 29). In one embodiment, CDR-H3 comprises SYGSYVSPLDY (SEQ ID NO: 30). In one embodiment, an antibody comprising these sequences (in combination as described herein) is humanized or human.

In one aspect, the invention provides an antibody comprising one, two, three, four, five or six CDRs, wherein each CDR comprises, consists or consists essentially of a sequence selected from the group consisting of SEQ ID NOs: 23, 24, 25, 26, 27, 28, and 29, and wherein SEQ ID NO:23 corresponds to an CDR-L1, SEQ ID NO:24 corresponds to an CDR-L2, SEQ ID NO:25 corresponds to an CDR-L3, SEQ ID NO:26 corresponds to an CDR-H1, SEQ ID NO:27 corresponds to an CDR-H2, and SEQ ID NOs:26, 27, or 28 corresponds to an CDR-H3. In one embodiment, an antibody of the invention comprises CDR-L1, CDR-L2, CDR-L3, CDR-H1, CDR-H2, and CDR-H3, wherein each, in order, comprises SEQ ID NO:23, 24, 25, 26, 27 and 29. In one embodiment, an antibody comprises CDR-L1, CDR-L2, CDR-L3, CDR-H1, CDR-H2, and CDR-H3, wherein each, in order, comprises SEQ ID NO:23, 24, 25, 26, 27 and 30.

Variant CDRs can have modifications of one or more residues within the CDR. In one embodiment, a CDR-L2 variant comprises 1-5 (1, 2, 3, 4 or 5) substitutions in any combination of the following positions: B1 (M or L), B2 (P, T, G or S), B3 (N, G, R or T), B4 (I, N or F), B5 (P, I, L or G), B6 (A, D, T or V) and B7 (R, I, M or G). In one embodiment, a CDR-H1 variant comprises 1-5 (1, 2, 3, 4 or 5) substitutions in any combination of the following positions: D3 (N, P, L, S, A, I), D5 (I, S or Y), D6 (G, D, T, K, R), D7 (F, H, R, S, T or V) and D9 (M or V). In one embodiment, a CDR-H2 variant comprises 1-4 (1, 2, 3 or 4) substitutions in any combination of the following positions: E7 (Y), E9 (I), E10 (I), E14 (T or Q), E15 (D, K, S, T or V), E16 (L), E17 (E, H, N or D) and E18 (Y, E or H). In one embodiment, a CDR-H3 variant comprises 1-5 (1, 2, 3, 4 or 5) substitutions in any combination of the following positions: F1 (T, S), F3 (R, S, H, T, A, K), F4 (G), F6 (R, F, M, T, E, K, A, L, W), F7 (L, I, T, R, K, V), F8 (S, A), F10 (Y, N) and F11 (Q, S, H, F). Letter(s) in parenthesis following each position indicates an illustrative substitution (i.e., replacement) amino acid; as would be evident to one skilled in the art, suitability of other amino acids as substitution amino acids in the context described herein can be routinely assessed using techniques known in the art and/or described herein. In one embodiment, a CDR-L1 comprises the sequence of SEQ ID NO:23. In one embodiment, F1 in a variant CDR-H3 is T. In one embodiment, F1 in a variant CDR-H3 is S. In one embodiment, F3 in a variant CDR-H3 is R. In one embodiment, F3 in a variant CDR-H3 is S. In one embodiment, F7 in a variant CDR-H3 is T. In one embodiment, an antibody comprises a variant CDR-H3 wherein F1 is T or S, F3 is R or S, and F7 is T.

In one embodiment, an antibody comprises a variant CDR-H3 wherein F1 is T, F3 is R and F7 is T. In one embodiment, an antibody comprises a variant CDR-H3 wherein F1 is S. In one embodiment, an antibody comprises a variant CDR-H3 wherein F1 is T, and F3 is R. In one embodiment, an antibody comprises a variant CDR-H3 wherein F1 is S, F3 is R and F7 is T. In one embodiment, an antibody comprises a variant CDR-H3 wherein F1 is T, F3 is S, F7 is T, and F8 is S. In one embodiment, an antibody comprises a variant CDR-H3 wherein F1 is T, F3 is S, F7 is T, and F8 is A. In some embodiments, said variant CDR-H3 antibody further comprises CDR-L1, CDR-L2, CDR-L3, CDR-H1 and CDR-H2 wherein each comprises, in order, the sequence depicted in SEQ ID NOs:1, 2, 3, 4 and 5. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody comprises a variant CDR-L2 wherein B6 is V. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:23, 25, 26, 27 and 28. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:23, 25, 26, 27 and 29. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:23, 25, 26, 27 and 30. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody of the invention comprises a variant CDR-H2 wherein E14 is T, E15 is K and E11 is E. In one embodiment, an antibody comprises a variant CDR-H2 wherein E11 is E. In some embodiments, said variant CDR-H3 antibody further comprises CDR-L1, CDR-L2, CDR-L3, CDR-H1, and CDR-H3 wherein each comprises, in order, the sequence depicted in SEQ ID NOs:23, 24, 25, 26, and 28. In some embodiments, said variant CDR-H2 antibody further comprises CDR-L1, CDR-L2, CDR-L3, CDR-H1, and CDR-H3, wherein each comprises, in order, the sequence depicted in SEQ ID NOs:23, 24, 25, 26, and 29. In some embodiments, said variant CDR-H2 antibody further comprises CDR-L1, CDR-L2, CDR-L3, CDR-H1, and CDR-H3, wherein each comprises, in order, the sequence depicted in SEQ ID NOs:23, 24, 25, 26 and 30. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In other embodiments, a c-met antibody specifically binds at least a portion of c-met Sema domain or variant thereof. In one example, an antagonist antibody specifically binds at least one of the sequences selected from the group consisting of LDAQT (SEQ ID NO: 33) (e.g., residues 269-273 of c-met), LTEKRKKRS (SEQ ID NO: 34) (e.g., residues 300-308 of c-met), KPDSAEPM (SEQ ID NO: 35) (e.g., residues 350-357 of c-met) and NVRCLQHF (SEQ ID NO: 36) (e.g., residues 381-388 of c-met). In one embodiment, an antagonist antibody specifically binds a conformational epitope formed by part or all of at least one of the sequences selected from the group consisting of LDAQT (SEQ ID NO: 33) (e.g., residues 269-273 of c-met), LTEKRKKRS (SEQ ID NO: 34) (e.g., residues 300-308 of c-met), KPDSAEPM (SEQ ID NO: 35) (e.g., residues 350-357 of c-met) and NVRCLQHF (SEQ ID NO: 36) (e.g., residues 381-388 of c-met). In one embodiment, an antagonist antibody specifically binds an amino acid sequence having at least 50%, 60%, 70%, 80%, 90%, 95%, 98% sequence identity or similarity with the sequence LDAQT (SEQ ID NO: 33), LTEKRKKRS (SEQ ID NO: 34), KPDSAEPM (SEQ ID NO: 35) and/or NVRCLQHF (SEQ ID NO: 36).

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816; Ridgeway, J et al, Prot Eng (1996) 9:617-21; Zhu Z et al. Prot Sci (1997) 6:781-8. For example, a hole mutation can be one or more of T366A, L368A and/or Y407V in an Fc polypeptide, and a knob mutation can be T366W.

### Anti-VEGF Antibodies and VEGF Antagonists

The VEGF antigen to be used for production of antibodies may be, e.g., the VEGF₁₆₅ molecule as well as other isoforms of VEGF or a fragment thereof containing the desired epitope. Other forms of VEGF useful for generating anti-VEGF antibodies of the invention will be apparent to those skilled in the art.

Human VEGF was obtained by first screening a cDNA library prepared from human cells, using bovine VEGF cDNA as a hybridization probe. Leung et al. (1989) Science, 246:1306. One cDNA identified thereby encodes a 165-amino acid protein having greater than 95% homology to bovine VEGF; this 165-amino acid protein is typically referred to as human VEGF (hVEGF) or VEGF₁₆₅. The mitogenic activity of human VEGF was confirmed by expressing the human VEGF cDNA in mammalian host cells. Media conditioned by cells transfected with the human VEGF cDNA promoted the proliferation of capillary endothelial cells, whereas control cells did not. Leung et al. (1989) Science, supra.

Although a vascular endothelial cell growth factor could be isolated and purified from natural sources for subsequent therapeutic use, the relatively low concentrations of the protein in follicular cells and the high cost, both in terms of effort and expense, of recovering VEGF proved commercially unavailing. Accordingly, further efforts were undertaken to clone and express VEGF via recombinant DNA techniques. (See, e.g., Ferrara, Laboratory Investigation 72:615-618 (1995), and the references cited therein).

VEGF is expressed in a variety of tissues as multiple homodimeric forms (121, 145, 165, 189, and 206 amino acids per monomer) resulting from alternative RNA splicing. VEGF₁₂₁ is a soluble mitogen that does not bind heparin; the longer forms of VEGF bind heparin with progressively higher affinity. The heparin-binding forms of VEGF can be cleaved in the carboxy terminus by plasmin to release a diffusible form(s) of VEGF. Amino acid sequencing of the carboxy terminal peptide identified after plasmin cleavage is Arg₁₁₀-Ala₁₁₁. Amino terminal “core” protein, VEGF (1-110) isolated as a homodimer, binds neutralizing monoclonal antibodies (such as the antibodies referred to as 4.6.1 and 3.2E3.1.1) and soluble forms of VEGF receptors with similar affinity compared to the intact VEGF₁₆₅ homodimer.

Several molecules structurally related to VEGF have also been identified recently, including placenta growth factor (PIGF), VEGF-B, VEGF-C, VEGF-D and VEGF-E. Ferrara and Davis-Smyth (1987) Endocr. Rev., supra; Ogawa et al. J. Biological Chem. 273:31273-31281 (1998); Meyer et al. EMBO J., 18:363-374 (1999). A receptor tyrosine kinase, Flt-4 (VEGFR-3), has been identified as the receptor for VEGF-C and VEGF-D. Joukov et al. EMBO. J. 15:1751 (1996); Lee et al. Proc. Natl. Acad. Sci. USA 93:1988-1992 (1996); Achen et al. (1998) Proc. Natl. Acad. Sci. USA 95:548-553. VEGF-C has been shown to be involved in the regulation of lymphatic angiogenesis. Jeltsch et al. Science 276:1423-1425 (1997).

Two VEGF receptors have been identified, Flt-1 (also called VEGFR-1) and KDR (also called VEGFR-2). Shibuya et al. (1990) Oncogene 8:519-527; de Vries et al. (1992) Science 255:989-991; Terman et al. (1992) Biochem. Biophys. Res. Commun. 187:1579-1586. Neuropilin-1 has been shown to be a selective VEGF receptor, able to bind the heparin-binding VEGF isoforms (Soker et al. (1998) Cell 92:735-45). Both Flt-I and KDR belong to the family of receptor tyrosine kinases (RTKs). The RTKs comprise a large family of transmembrane receptors with diverse biological activities. At present, at least nineteen (19) distinct RTK subfamilies have been identified. The receptor tyrosine kinase (RTK) family includes receptors that are crucial for the growth and differentiation of a variety of cell types (Yarden and Ullrich (1988) Ann. Rev. Biochem. 57:433-478; Ullrich and Schlessinger (1990) Cell 61:243-254). The intrinsic function of RTKs is activated upon ligand binding, which results in phosphorylation of the receptor and multiple cellular substrates, and subsequently in a variety of cellular responses (Ullrich & Schlessinger (1990) Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response. (e.g., cell division, differentiation, metabolic effects, changes in the extracellular microenvironment) see, Schlessinger and Ullrich (1992) Neuron 9:1-20. Structurally, both Flt-1 and KDR have seven immunoglobulin-like domains in the extracellular domain, a single transmembrane region, and a consensus tyrosine kinase sequence which is interrupted by a kinase-insert domain. Matthews et al. (1991) Proc. Natl. Acad. Sci. USA 88:9026-9030; Terman et al. (1991) Oncogene 6:1677-1683.

Anti-VEGF antibodies that are useful in the methods of the invention include any antibody, or antigen binding fragment thereof, that bind with sufficient affinity and specificity to VEGF and can reduce or inhibit the biological activity of VEGF. An anti-VEGF antibody will usually not bind to other VEGF homologues such as VEGF-B or VEGF-C, nor other growth factors such as PlGF, PDGF, or bFGF.

In certain embodiments of the invention, the anti-VEGF antibodies include, but are not limited to, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599. In one embodiment, the anti-VEGF antibody is “Bevacizumab (BV)”, also known as “rhuMAb VEGF” or “AVASTIN®”. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1.

Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, and US Patent Application Publication US2009-0142343, the content of these patent applications are expressly incorporated herein by reference. For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, I83 and Q89.

In one embodiment of the invention, the anti-VEGF antibody has a heavy chain variable region comprising the following amino acid sequence:

(SEQ ID NO: 37)
EVQLVESGGG LVQPGGSLRL SCAASGYTFT NYGMNWVRQA
PGKGLEWVGWINTYTGEPTY AADFKRRFTF SLDTSKSTAY
LQMNSLRAED TAVYYCAKYPHYYGSSHWYF DVWGQGTLVT
VSS

and a light chain variable region comprising the following amino acid sequence:

(SEQ ID NO: 38)
DIQMTQSPSS LSASVGDRVT ITCSASQDIS NYLNWYQQKP
GKAPKVLIYFTSSLHSGVPS RFSGSGSGTD FTLTISSLQP
EDFATYYCQQ YSTVPWTFGQGTKVEIKR.

A “G6 series antibody” according to this invention, is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, I83 and Q89.

A “B20 series antibody” according to this invention is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and US Patent Application Publication US2009-0142343, the content of these patent applications are expressly incorporated herein by reference. In one embodiment, the B20 series antibody binds to a functional epitope on human VEGF comprising residues F17, M18, D19, Y21, Y25, Q89, I91, K101, E103, and C104.

A “functional epitope” according to this invention refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC50 mutant VEGF/IC50 wild-type VEGF) of the antibody will be greater than 5 (see Example 2 of WO2005/012359). In one embodiment, the relative affinity ratio is determined by a solution binding phage displaying ELISA. Briefly, 96-well Maxisorp immunoplates (NUNC) are coated overnight at 4° C. with an Fab form of the antibody to be tested at a concentration of 2 ug/ml in PBS, and blocked with PBS, 0.5% BSA, and 0.05% Tween20 (PBT) for 2 h at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′,5,5′-tetramethylbenzidine (TMB, Kirkegaard & Perry Labs, Gaithersburg, Md.) substrate for approximately 5 min, quenched with 1.0 M H3PO4, and read spectrophotometrically at 450 nm. The ratio of IC50 values (IC50, ala/IC50, wt) represents the fold of reduction in binding affinity (the relative binding affinity).

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules, or fragments thereof, that specifically bind to VEGF can be used in the methods of the invention to bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the flt-1 or KDR receptor), that is capable of binding to and inhibiting the biological activity of VEGF. In certain embodiments, the chimeric VEGF receptor proteins of the invention consist of amino acid sequences derived from only two different VEGF receptor molecules; however, amino acid sequences comprising one, two, three, four, five, six, or all seven Ig-like domains from the extracellular ligand-binding region of the flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include, e.g., soluble Flt-1/Fc, KDR/Fc, or FLt-1/KDR/Fc (also known as VEGF Trap). (See for example PCT Application Publication No. WO97/44453)

A soluble VEGF receptor protein or chimeric VEGF receptor proteins of the invention includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

### Therapies

The present invention features the combination use of an anti-c-met antibody and a chemotherapeutic (e.g., a taxane such as paclitaxel) as part of a specific treatment regimen intended to provide a beneficial effect from the combined activity of these therapeutic agents. The present invention also features the combination use of an anti-c-met antibody, an anti-VEGF antibody, and a chemotherapeutic (e.g., a taxane such as paclitaxel) as part of a specific treatment regimen intended to provide a beneficial effect from the combined activity of these therapeutic agents. The beneficial effect of the combination includes, but is not limited to, pharmacokinetic or pharmacodynamic co-action resulting from the combination of therapeutic agents.

In one embodiment, the invention provides methods for the treatment of breast cancer, comprising administering to an ER-negative, PR-negative, and HER2-negative (ER-, PR-, and HER2-; or triple-negative) metastatic breast cancer patient an anti-c-met antibody (e.g., MetMAb) administered at a dose of 10 mg/kg on Day 1 and Day 15 of a 28-day cycle and paclitaxel administered at a dose of 90 mg/m² by IV infusion on Day 1, Day 8, and Day 15 of the 28-day cycle.

In one embodiment, the invention provides methods for the treatment of breast cancer, comprising administering to an ER-negative, PR-negative, and HER2-negative (ER-, PR-, and HER2-; or triple-negative) metastatic breast cancer patient an anti-c-met antibody (e.g., MetMAb) administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle.

The present invention also features the use of an anti-c-met antibody as part of a specific treatment regimen intended to provide a beneficial effect from the activity of this therapeutic agent. Thus, in one aspect, the invention provides methods of treating a cancer in a subject, comprising administering to the subject an anti-c-met antibody at a dose of about 10 mg/kg every two weeks.

In another aspect, the invention provides methods of treating a cancer in a subject, comprising administering to the subject (a) an anti-c-met antibody at a dose of about 10 mg/kg every two weeks; and (b) a VEGF antagonist (such as an anti-VEGF antibody).

Although the methods of the present invention may be performed in the absence of any other means of cancer therapy, e.g. in the absence of a further therapeutic agent, including chemotherapeutic agents, the methods may optionally comprise the administration of a further therapeutic agent selected from the group consisting of chemotherapeutic agent, a different anti-c-met antibody, a different anti-VEGF antibody, antibody directed against a tumor associated antigen, anti-hormonal compound, cardioprotectant, cytokine, anti-angiogenic agent, tyrosine kinase inhibitor, COX inhibitor, non-steroidal anti-inflammatory drug, farnesyl transferase inhibitor, antibody that binds oncofetal protein CA 125, Raf or ras inhibitor, liposomal doxorubicin, topotecan, a different taxane, a medicament that treats nausea, a medicament that prevents or treats skin rash or standard acne therapy, a medicament that treats or prevents diarrhea, a body temperature-reducing medicament, and a hematopoietic growth factor.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies and other therapeutic agents would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Where the inhibitor is an antibody, the antibody can be an immunoconjugate. Preferably, the conjugated inhibitor and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases and DNA endonucleases.

Depending on the type and severity of the disease, about 1 μg/kg to 100 mg/kg (e.g., 0.1-20 mg/kg) of VEGF-specific antagonist is an initial candidate dosage for administration to the patient, whether, for example, by one or more separate administrations, or by continuous infusion. A typical daily dosage might range from about 1 μg/kg to about 100 mg/kg or more, depending on the factors mentioned above. Particularly desirable dosages include, for example, 5 mg/kg, 7.5 mg/kg, 10 mg/kg, and 15 mg/kg. For repeated administrations over several days or longer, depending on the condition, the treatment is sustained until the cancer is treated, as measured by the methods described above or known in the art. However, other dosage regimens may be useful. In one example, if the VEGF-specific antagonist is an antibody, the antibody of the invention is administered once every week, every two weeks, or every three weeks, at a dose range from about 5 mg/kg to about 15 mg/kg, including but not limited to 5 mg/kg, 7.5 mg/kg, 10 mg/kg or 15 mg/kg. The progress of the therapy of the invention is easily monitored by conventional techniques and assays.

In one example, the anti-c-met antibody (e.g., MetMAb) administered at a dose of 10 mg/kg on Day 1 and Day 15 of a 28-day cycle. In another example, an anti-c-met antibody (e.g., MetMAb) administered at a dose of 15 mg/kg every three weeks. In some embodiments, the anti-c-met antibody is administered in an amount sufficient to achieve a serum trough concentration at or above 15 micrograms/ml. In some embodiments, the anti-c-met antibody is administered at a total dose of about 15 mg/kg over a three week period.

The duration of therapy will continue for as long as medically indicated or until a desired therapeutic effect (e.g., those described herein) is achieved.

In some embodiments, the patient herein is subjected to a diagnostic test e.g., prior to and/or during and/or after therapy. Generally, if a diagnostic test is performed, a sample may be obtained from a patient in need of therapy. Where the subject has cancer, the sample may be a tumor sample, or other biological sample, such as a biological fluid, including, without limitation, blood, urine, saliva, ascites fluid, or derivatives such as blood serum and blood plasma, and the like.

In some embodiments, the pattern of expression of biomarkers such as ER, PR, HER2, EGFR, and cytokeratins can be used to stratify breast cancers into distinct subtypes. In some embodiments, HER2 status will be identified by immunohistochemistry and/or fluorescence in-situ hybridization (FISH) assays. In some embodiments, patients who meet any of the following will be categorized as HER2 negative:

IHC negative (IHC 0 or 1+ score)

IHC positive (IHC 2+ or 3+ score; definition may vary by site) and FISH negative (HER2/CEP17 ratio <1.8 or HER2 gene copies/nucleus <4)

FISH negative (HER2/CEP17 ratio <1.8)

FISH negative (HER2 gene copies/nucleus <4)

ER and PR status may be determined.

In some embodiments, the subject's cancer expresses c-met. Methods for determining c-met expression are known in the art, e.g., IHC and FISH. Using IHC methods, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody.

In some embodiments, serum from a subject expresses IL8, in some embodiments, supranormal levels of IL8. In some embodiments, serum from a subject expresses greater than about 150 pg/ml of IL8, or in some embodiments, greater than about 50 pg/ml IL8. In some embodiments, serum from a subject expresses greater than about 10 pg/ml, 20 pg/ml, 30 pg/ml or more of IL8. Methods for determining IL8 serum concentration are known in the art.

In some embodiments, serum from a subject expresses HGF, in some embodiments, supranormal levels of HGF. In some embodiments, serum from a subject expresses greater than about 5,000, 10,000, or 50,000 pg/ml of HGF.

In some embodiments, decreased mRNA or protein expression in a sample, e.g., from a tumor or serum in a patient treated with a c-met antagonist, and in some embodiments, further treated with a VEGF antagonist and a taxane (such as paclitaxel), is prognostic, e.g. for response to treatment or for c-met antagonist activity. In some embodiments, decreased expression of several angiogenic factor, such as interleukin 8 (IL8), vascular endothelial cell growth factor A (VEGFA), EPH receptor A2 (EphA2), Angiopoietin-like4 (Angptl4), and Ephrin B2 (EFNB2), is prognostic, e.g. for response to treatment or for c-met antagonist activity. Decrease in expression may be determined relative to an untreated sample or with reference to a normal value or relative to the patient's expression level prior to treatment with the c-met antagonist (or treatment with c-met antagonist, VEGF antagonist and a taxane).

In some embodiments, decreased HGF or IL8 expression in a sample, e.g., from a tumor or serum in a patient is prognostic, e.g. for response to treatment or for c-met antagonist (and in some embodiments for response to c-met antagonist, VEGF antagonist and taxane) activity. In one embodiment, a greater than 50% decrease or a greater than 70% decrease (e.g., relative to IL8 expression level in the patient prior to treatment) in IL8 expression in serum indicates response to treatment. Decrease in expression may be determined relative to an untreated sample or with reference to a normal value or relative to the patient's expression level prior to treatment with the c-met antagonist (or treatment with c-met antagonist and VEGF antagonist).

In some embodiments, increased mRNA or protein expression in a sample, e.g., from a tumor or serum in a patient treated with a c-met antagonist, and in some embodiments, further treated with a VEGF antagonist, is prognostic, e.g. for response to treatment or for c-met antagonist (and in some embodiments for response to c-met antagonist, VEGF antagonist and taxane) activity. Decrease in expression may be determined relative to an untreated sample or with reference to a normal value or relative to the patient's expression level prior to treatment with the c-met antagonist (or treatment with c-met antagonist and VEGF antagonist)

In some embodiments, FDG-PET imaging is prognostic, e.g. for response to treatment or for c-met antagonist activity).

The sample herein may be a fixed sample, e.g. a formalin fixed, paraffin-embedded (FFPE) sample, or a frozen sample.

### Formulations

Pharmaceutical formulations of an antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20O, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

In one embodiment, bevacizumab is supplied for therapeutic uses in 100 mg and 400 mg preservative-free, single-use vials to deliver 4 ml or 16 ml of bevacizumab (25 mg/ml). The 100 mg product is formulated in 240 mg α,α-trehalose dehydrate, 23.2 mg sodium phosphate (monobasic, monohydrate), 4.8 mg sodium phosphate (dibasic, anhydrous), 1.6 mg polysorbate 20, and Water for Injection, USP. The 400 mg product is formulated in 960 mg α,α-trehalose dehydrate, 92.8 mg sodium phosphate (monobasic, monohydrate), 19.2 mg sodium phosphate (dibasic, anhydrous), 6.4 mg polysorbate 20, and Water for Injection, USP. See also the label for bevacizumab. Bevacizumab is currently available commercially. The formulation herein may also contain more than one active compound as necessary for the particular indication being treated, preferably those with complementary activities that do not adversely affect each other. Such molecules are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Efficacy

The main advantage of the treatment of the invention is the ability of producing marked anti-cancer effects in a human patient without causing significant toxicities or adverse effects, so that the patient benefited from the treatment overall. The efficacy of the treatment of the invention can be measured by various endpoints commonly used in evaluating cancer treatments, including but not limited to, tumor regression, tumor weight or size shrinkage, time to progression, duration of survival, progression free survival, overall response rate, duration of response, and quality of life. The therapeutic agents of the invention may cause inhibition of metastatic spread without shrinkage of the primary tumor, or may simply exert a tumouristatic effect. Because the anti-angiogenic agents used in the invention target the tumor vasculature and not necessarily the neoplastic cells themselves, they represent a unique class of anticancer drugs, and therefore may require unique measures and definitions of clinical responses to drugs. For example, tumor shrinkage of greater than 50% in a 2-dimensional analysis may be used as a cut-off for declaring a response. Accordingly, novel approaches to determining efficacy of a therapy can be optionally employed, including for example, measurement of plasma or urinary markers of angiogenesis and measurement of response through radiological imaging.

### Antibody Preparation

In a further aspect, an antibody according to any of the above embodiments may incorporate any of the features, singly or in combination, as described in Sections 1-7 below:

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, one-armed antibodies, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Other monovalent antibody forms are described in, e.g., WO2007048037, WO2008145137, WO2008145138, and WO2007059782. One-armed antibodies are described, e.g., in WO2005/063816. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which CDRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al. Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for an antigen and the other is for any other antigen. In certain embodiments, bispecific antibodies may bind to two different epitopes of an antigen. Bispecific antibodies may also be used to localize cytotoxic agents to cells which express an antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to an antigen as well as another, different antigen (see, US 2008/0069820, for example).

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants

In certain embodiments, antibody variants having one or more amino acid substitutions are provided. Sites of interest for substitutional mutagenesis include the CDRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Tip, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Methods and Compositions

Antibodies may be produced using recombinant methods and compositions, e.g., as described in U.S. Pat. No. 4,816,567. In one embodiment, isolated nucleic acid encoding an antibody is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. Production of a one-armed antibody is described, e.g., in WO2005/063816.

In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Immunoconjugates

The invention also provides immunoconjugates comprising an antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

The following are examples of the methods and compositions of the invention. It is understood that various other embodiments may be practiced, given the general description provided above.

### EXAMPLES

### Example 1

### A Phase I Open-Label Dose-Escalation Study of the Safety and Pharmacology of MetMAb, a monovalent antagonist antibody to the receptor c-met, Administered Intravenously in Patient with Locally Advanced or Metastatic Solid Tumors

This example describes a Phase I, open-label, dose-escalation study of MetMAb administered by IV infusion every 3 weeks (Q3W) in patients with advanced solid malignancies that are refractory to or for which there is no standard of care. This dose-escalation trial tested the combination of MetMAb, at two different doses, with bevacizumab at 15 mg/kg IV Q3W.

Study Design.

Bevacizumab (15 mg/kg Q3W) was dosed with one of two doses of MetMAb (10 or 15 mg/kg Q3W). In the first cohort, 3 patients received MetMAb (10 mg/kg) and bevacizumab (15 mg/kg) IV once every 3 weeks. In the second cohort, 6 patients received MetMAb (15 mg/kg, the recommended Phase II dose) and bevacizumab (15 mg/kg) IV once every 3 weeks.

Study objectives. The objectives of this study included determining the safety and tolerability of MetMAb in combination with bevacizumab at 15 mg/kg administered intravenously every 3 weeks.

Exclusion Criteria:
Patients of childbearing potential must be using effective contraception.
Inability to comply with study and follow-up procedures.
Inadequately controlled hypertension (defined as systolic blood pressure >150 mmHg

and/or diastolic blood pressure >100 mmHg).
Prior history of hypertensive crisis or hypertensive encephalopathy.
New York Heart Association (NYHA) Class II or greater CHF.
History of myocardial infarction or unstable angina within 6 months prior to Day 1.
History of stroke or transient ischemic attack (TIA) within 6 months prior to study enrollment.
Significant vascular disease (e.g., aortic aneurysm requiring surgical repair or recent peripheral arterial thrombosis) within 6 months prior to Day 1.
History of hemoptysis (≧½ teaspoon of bright red blood per episode) within 1 month

prior to Day 1.
Evidence of bleeding diathesis or significant coagulopathy (in the absence of therapeutic anticoagulation).
Major surgical procedure, open biopsy, or significant traumatic injury within 28 days

prior to Day 1 or anticipation of need for major surgical procedure during the course of

the study.
Core biopsy or other minor surgical procedure, excluding placement of a vascular access

device, within 7 days prior to Day 1.
History of abdominal fistula or gastrointestinal perforation within 6 months prior to Day 1.
Serious, non-healing wound, active ulcer, or untreated bone fracture.
Proteinuria at screening, as demonstrated by a UPC ratio of ≧1.0 at screening.
Known hypersensitivity to any component of bevacizumab.
Pregnancy (positive pregnancy test) or lactation.

Trial Drugs

MetMAb was supplied as either a lyophilized powder or as a sterile liquid. MetMAb provided as a lyophilized powder (400 mg) was supplied in a single-use 50-cc vial for the Phase I study. The solution for reconstitution was sterile water for injection and the reconstitution volume was 20.0 mL to yield a final concentration of 20 mg/mL MetMAb in 10 mM histidine succinate, 106 mM (4%) trehalose dihydrate, 0.02% polysorbate 20, pH 5.7. MetMAb provided as a sterile liquid was supplied in a single-use 15-cc vial. Each vial contained 600 mg of MetMAb in 10 ml at a concentration of 60 mg/ml in 10 mM histidine acetate, 120 mM trehalose, 0.02% polysorbate 20, pH 5.4. The total dose of MetMAb for each patient depended on dose level assignment and the patient's weight on, or within 14 days prior to, Day 1 of Cycle 1.

Bevacizumab was supplied by Genentech, Inc., as a clear to slightly opalescent, sterile liquid ready for parenteral administration. Each 400-mg or 100-mg (25 mg/mL) glass vial contained bevacizumab with a vehicle consisting of sodium phosphate, trehalose, polysorbate 20, and Sterile Water for Injection, USP. Vials contained no preservative and were for single use only. The bevacizumab dose was based on the patient's weight at screening and remained the same throughout the study.

Results

In this Phase Ib study, the combination of MetMAb with bevacizumab was generally well tolerated at all doses tested. Patient demographics are shown in Table 2.

TABLE 2
Patient Demographics (n = 9)

| Age (yr) | (n = 9) | |
|---|---|---|
| Mean (SD) | 54.9 | (14.9) |
| Median | 46.0 | |
| Range | 42-80 | |
| Sex, n(%) | | |
| Female | 7 | (77.8) |
| Male | 2 | (22.2) |

Number of prior therapy regiments for patients in this trial and in a previously described phase 1a dose escalation trial (Salgia R et al. Complete results from a Phase Ia dose escalation and dose expansion study of single agent MetMab, a monovalent antagonist antibody to the receptor met, administered intravenously in patients with locally advanced or metastatic solid tumors. AACR 2010, Abstract 2774; see also WO2010/045345) are shown in Table 3.

TABLE 3
number of prior therapy regiments* (n = 43)

| | |
|---|---|
| 1 | 4 |
| 2 | 9 |
| 3 | 10 |
| greater than or equal to 4 | 20 |

*Includes chemotherapy, radiotherapy, and targeted/biologic therapy.

FIG. 5 depicts patient diagnosis, treatment cohort and administered cycles for this trial (“MetMAb+Bev”) and for a previously described Phase 1 trial (“MetMAb”) (Salgia R, et al. AACR 2010, Abstract 2774).

Pharmacokinetic analysis of this trial in combination with a previously described Phase 1 trial (Salgia R, et al. AACR 2010, Abstract 2774) showed that the terminal half-life of MetMAb is 11 days and clearance is ˜7 (+/2.0) mL/day/kg. This clearance rate is approximately 2 times faster than that of traditional bivalent antibodies. MetMAb had no apparent PK interaction with bevacizumab. 12% of patients were positive for anti-treatment antibodies (ATAs) to MetMAb with all ATA responses directed primarily toward the framework of MetMAb (assay validated with a 5% untreated false positive rate; (assay sensitivity was 143 ng/mL; minimum reportable titer value of 1.4).

Safety results are shown in Table 4.

TABLE 4
All Drug-Related Grade 1 or 2 Adverse Events (>5%) and
All Drug-Related Grade 3 Adverse Events

| | Stage 1 and 2 | | | | Stage 3 (MetMab + | | |
|---|---|---|---|---|---|---|---|
| | (n = 34) | | | | Bev) (n = 9) | | |
| | Gr 1 or 2 | | Gr 3* | | Gr 1 or 2 | | Gr 3* |
| Any adverse | 15 | (44.1) | 7 | (20.6) | | | |
| event, n(%) | | | | | | | |
| Fatigue | 13 | (38.2) | 0 | | 2 | (22.2) | 0 |
| Edema, peripheral | 6 | (17.6) | 3 | (8.8) | 2 | (22.2) | 0 |

| Hypoalbuminemia | 4 | (11.8) | 0 | 1 | (11.1) | 0 |
|---|---|---|---|---|---|---|
| Nausea | 4 | (11.8) | 0 | 1 | (11.1) | 0 |
| Vomiting | 3 | (8.8) | 0 | | | |
| Weight Increased | | | | 2 | (22.2) | 0 |
| Anorexia | 3 | (8.8) | 0 | 1 | (11.1) | 0 |

| | | | |
|---|---|---|---|
| Muscle spasms | 3 | (8.8) | 0 |

| | | | |
|---|---|---|---|
| Abdominal pain | 0 | 1 | (2.9) |
| AST increased | 0 | 1 | (2.9) |
| Pyrexia** | 0 | 1 | (2.9) |
| Hyponatremia | 0 | 1 | (2.9) |

| | | | |
|---|---|---|---|
| Hypoalbuminemia | 1 | (11.1) | 0 |
| Hemoptysis** | 1 | (11.1) | 0 |
| Hypertension | 2 | (22.2) | 0 |

*There were no Grade 4 events;
**Dose-limiting toxicity;
AST = aspartate amonotransferase

No Grade 3-5 drug-related toxicities were observed. One dose-limiting toxicity (DLT) of Grade 1 hemoptysis (<1 tsp) was observed in one patient (gastric cancer with pulmonary metastasis, showing central necrosis at time of event) in the second cohort. Grade 2 drug-related toxicities included peripheral edema and hypoalbuminemia. The most frequently observed toxicities (>30%) included fatigue (56%), edema (33%), and increased weight (33%).

FIG. 6 depicts change of tumor burden from baseline with best response, all patients for the present study (“stage 3”) and a previously reported Phase 1 study (“phase 1 and 2”) (Salgia R, et al. AACR 2010, Abstract 2774). The best response was stable disease, with 3 patients receiving ≧6 cycles.

Conclusion: The combination of MetMAb and bevacizumab is generally safe and well tolerated at the recommended dose of 15 mg/kg IV q3W for each agent. No drug-related Grade 4 toxicities were observed.

### Example 2

### A Phase II Study Evaluating the Safety and Efficacy of MetMab in Combination with Paclitaxel and Bevacizumab in Patients with Metastatic, Triple-Negative Breast Cancer (OAM4861g)

Metastatic breast cancer is the most common invasive malignancy in females, and the second most common cause of cancer death in women, with the majority of patients succumbing to their disease within 2 years of diagnosis (Greenberg et al. 1996). According to the Surveillance, Epidemiology and End Results (SEER) database, over 192,000 women were diagnosed with and greater than 40,000 women died of cancer of the breast in 2009 in the United States (SEER 2009). The lifetime probability of developing invasive breast cancer is one in eight.

The treatment algorithm for patients with metastatic breast cancer is based on several factors that include clinical, pathologic, and histologic characteristics such as human epidermal growth factor 2 (HER2) amplification, hormone receptor (ER, PR) status, prior response to and/or failure of hormonal agents, number and specific sites of metastatic disease, and treatment history in both the metastatic and adjuvant settings. Numerous cytotoxic chemotherapy agents have shown activity in metastatic breast cancer, including anthracyclines, taxanes, gemcitabine, capecitabine, and vinorelbine. The response rates and progression-free intervals seen with these agents vary, depending on the extent/type of prior therapy and extent of metastatic disease. In general, anthracycline-based combination therapy and taxanes (paclitaxel and docetaxel) are believed to show the greatest activity. Given the high use of regimens containing anthracyclines in the adjuvant setting combined with the limitation with repeat courses of anthracyclines, taxanes are now the most commonly used agent for patients with locally recurrent or metastatic disease.

Triple-negative breast cancers are more likely to have aggressive features, such as high proliferative rate, and exhibit an invasive phenotype. Patients with metastatic triple negative breast cancer exhibit a poor clinical outcome and a median survival of less than one year. Most, but not all, basal-like breast cancers are triple-negative by IHC test, and, as a result, triple-negative status may be used as a histopathological definition of basal-like breast cancer. All current basal-like breast cancer trials presently registered with the National Cancer Institute (NCI) use the biomarker triplet (ER, PR and HER2) to identify eligible patients.

New treatments directed at delaying disease progression while avoiding systemic toxicity would represent a significant advance in the treatment of these patients.

An analysis of a large panel of breast cancer cell lines showed that Met is expressed selectively in basal lines, relative to luminal or HER2-positive cell lines, suggesting that Met expression and activation may be important for initiation and progression of triple-negative breast cancer.

This Example describes a randomized, Phase II, double-blind, multicenter, placebo-controlled trial designed to preliminarily estimate the efficacy and evaluate the safety and tolerability of MetMAb administered in combination with paclitaxel, and MetMAb administered in combination with bevacizumab+paclitaxel versus placebo+bevacizumab+paclitaxel in patients with metastatic or locally recurrent, triple-negative breast cancer who either have not received treatment (first-line) or have progressed after one conventional cytotoxic chemotherapy regimen (second-line), with the regimen defined as single-agent chemotherapy administered prior to or during disease progression or a pre-specified combination or sequence of cytotoxic agents administered in the first-line setting. Approximately 180 patients (approximately 120 patients who have not received previous treatment and 60 patients receiving second-line therapy) from approximately 40 multinational sites will be randomized in a 1:1:1 ratio to the three treatment groups. All patients must have histologically confirmed triple-negative adenocarcinoma of the breast, with measurable or non-measurable metastatic or locally recurrent disease.

Objectives:

The primary objective of this study is to estimate the clinical benefit of MetMAb+bevacizumab+paclitaxel and MetMAb+placebo+paclitaxel relative to placebo+bevacizumab+paclitaxel, as measured by investigator-assessed progression free survival, in patients with metastatic or locally recurrent, triple-negative breast cancer who have received no prior systemic therapy or have progressed following first-line therapy. PFS, defined as the time from randomization to disease progression or relapse (as assessed by the site radiologist and/or investigator, using Response Evaluation Criteria In Solid Tumors [RECIST], Version 1.1) or death on study from any cause (defined as death within 30 days of the last study treatment), whichever occurs first.

The secondary objectives of this study include:

To estimate the clinical benefit of MetMAb+bevacizumab+paclitaxel and MetMAb+placebo+paclitaxel relative to placebo+bevacizumab+paclitaxel, as measured by investigator-assessed progression-free survival, in patients with metastatic or locally recurrent, triple-negative breast cancer who have received no prior systemic therapy or have progressed following first-line therapy.

To determine the overall response rate and duration of response of MetMAb+bevacizumab+paclitaxel and MetMAB+placebo+paclitaxel relative to placebo+bevacizumab+paclitaxel in patients with metastatic or locally recurrent, triple-negative breast cancer who have received no prior systemic therapy or have progressed following first-line therapy. Objective response is defined as a complete or partial response maintained ≧4 weeks (as assessed by the site radiologist and/or investigator, using RECIST). Duration of response is defined as the time from initial complete or partial response to disease progression (as assessed by the site radiologist and/or investigator, using RECIST) or death on study from any cause (defined as death within 30 days of the last study treatment), whichever occurs first.

To evaluate overall survival benefit of MetMAb+bevacizumab+paclitaxel and MetMAB+placebo+paclitaxel relative to placebo+bevacizumab+paclitaxel in patients with metastatic or locally recurrent, triple-negative breast cancer who have received no prior systemic therapy or have progressed following first-line therapy. Overall survival is defined as the time from randomization to death from any cause.

To characterize the safety and tolerability of MetMAb+bevacizumab+paclitaxel and MetMAB+placebo+paclitaxel relative to placebo+bevacizumab+paclitaxel

To evaluate drug exposure of MetMab, bevacizumab and paclitaxel.

Inclusion Criteria in the Study Include the Following:

Signed Informed Consent Form.

Age ≧18 years.

Eastern Cooperative Oncology Group (ECOG) Performance Status of 0 or 1. Histologically confirmed ER-, PR-, and HER2-negative (triple-negative) adenocarcinoma of the breast, with measurable or non-measurable metastatic or locally recurrent disease.

For women of childbearing potential, use of accepted and effective method of contraception.

Ability and capacity to comply with study and follow-up procedures.

Exclusion Criteria in the Study Includes the Following:

Prior therapy with two or more regimens for metastatic breast cancer.

Any systemic anti-cancer therapy within 3 weeks prior to Day 1 of Cycle 1.

Major surgical procedure (except CNS surgery), open biopsy, or significant traumatic injury within 30 days prior to Day 1 of Cycle 1, or anticipation of need for major surgical procedure during the course of the study.

Minor surgical procedures, such as fine-needle aspirations or core biopsies, within 7 days prior to Day 1 of Cycle 1.

Prior therapy with a taxane for metastatic breast cancer.

Prior therapy with bevacizumab, sorafenib, sunitinib, or other putative VEGF pathway—targeted therapy following diagnosis of breast cancer.

Prior exposure to experimental treatment targeting either the HGF or MET pathways.

Prior therapy with hormones and/or trastuzumab.

Known brain or other CNS metastases, except for treated brain metastasis.

Uncontrolled hypertension defined by systolic pressure >150 mmHg and/or diastolic pressure >100 mmHg, with or without anti-hypertensive medication.

Patients with initial blood pressure elevations are eligible if initiation or adjustment of anti-hypertensive medication lowers blood pressure to meet entry criteria.

Unstable angina.

Prior history of hypertensive crisis or hypertensive encephalopathy.

New York Heart Association Grade ≧II congestive heart failure.

History of myocardial infarction within 6 months prior to Day 1 of Cycle 1.

History of stroke or transient ischemic attack within 6 months prior to Day 1 of Cycle 1.

Clinically significant peripheral vascular disease (e.g., aortic aneurysm requiring surgical repair or recent peripheral arterial thrombosis) within 6 months prior to Day 1 of Cycle 1.

Evidence of bleeding diathesis or coagulopathy.

History of abdominal fistula, gastrointestinal perforation, or intra-abdominal abscess within 6 months prior to Day 1 of Cycle 1.

History of anaphylactic reaction to monoclonal antibody therapy not controlled with treatment pre-medication.

History of hemoptysis (≧½ teaspoon of bright red blood per episode) within 1 month prior to Day 1 of Cycle 1.

Known hypersensitivity to any component of bevacizumab.

Serious non-healing wound, active ulcer, or untreated bone fracture.

Trial drugs. MetMAb is a known recombinant, humanized, monovalent monoclonal antibody directed against c-met. MetMAb will be supplied as a sterile liquid in a single-use, 15-cc vial. Each vial contains 600 mg of MetMAb in 10 mL at a concentration of 60 mg/mL in 10 mM histidine acetate, 120 mM trehalose, and 0.02% polysorbate 20, pH 5.4.

Bevacizumab is a clear to slightly opalescent, colorless to pale brown, sterile liquid concentrate for solution for IV infusion. Bevacizumab will be supplied in either 5-mL (100-mg) or 20-mL (400-mg) glass vials containing 4 mL or 16 mL of bevacizumab, respectively (25 mg/mL for either vial). Vials contain bevacizumab with phosphate, trehalose, polysorbate 20, and Sterile Water for Injection (SWFI), USP. Vials contain no preservative and are suitable for single use only.

Refer to the TAXOL® Package Insert for information on the formulation for paclitaxel.

Placebo will consist of 250 cc 0.9% NSS (saline IV solution, 0.9%).

Study Treatment:

Pharmacokinetic modeling showed that a MetMAb dose of 10 mg/kg every 2 weeks is predicted to result in equivalent exposure relative to a MetMAb dose of 15 mg/kg every 3 weeks.

MetMAb and bevacizumab will each be administered at a dose of 10 mg/kg by IV infusion every 2 weeks, on Day 1 and Day 15 of each 28-day cycle. Paclitaxel will be administered at a dose of 90 mg/m² by IV infusion on Day 1, Day 8, and Day 15 of each 28-day cycle. The order of administration of the drugs when all three are administered on the same day is the following: 1) paclitaxel, 2) bevacizumab, and 3) MetMAb/placebo.

The dose of MetMAb will be based on the patient's weight at screening or baseline and will remain the same throughout the study. The dose of bevacizumab will be based on the patient's weight at screening and will remain the same throughout the study. Calculation of body surface area for the purposes of dosing of paclitaxel should be made according to the prescribing information.

Results

Administration of (1) MetMAb at 10 mg/kg (e.g., based on subject's weight at Day 1 or at screening) at Day 1 and Day 15 of a 28-day cycle; and (2) paclitaxel at a dose of 90 mg/m² by IV infusion on Day 1, Day 8, and Day 15 of a 28-day cycle to triple-negative metastatic breast cancer patients extended time to disease progression (TTP) and/or progression-free survival, and survival. Administration of (1) MetMAb at 10 mg/kg (e.g., based on subject's weight at Day 1 or at screening) at Day 1 and Day 15 of a 28-day cycle; (2) bevacizumab at a dose of 10 mg/kg by IV infusion every 2 weeks, on Day 1 and Day 15 of a 28-day cycle, and (3) paclitaxel at a dose of 90 mg/m² by IV infusion on Day 1, Day 8, and Day 15 of a 28-day cycle to triple-negative metastatic breast cancer patients extended time to disease progression (TTP) and/or progression-free survival, and survival.

Although the foregoing invention has been described in some detail by way of illustration and example for purposes of clarity of understanding, the descriptions and examples should not be construed as limiting the scope of the invention.

1. A method for the treatment of breast cancer, comprising administering to an estrogen receptor (ER)-negative, progesterone receptor (PR)-negative and HER2-negative (collectively, triple-negative) metastatic breast cancer patient an effective amount of an anti-c-met antibody and a taxane.

2. The method of claim 1, further comprising administering to the patient an effective amount of an anti-VEGF antibody.

3. A method for the treatment of breast cancer, comprising administering to an ER-negative, PR-negative, and HER2-negative metastatic breast cancer patient an anti-c-met antibody administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

4. The method of claim 3, further comprising administering anti-VEGF antibody at a dose of 10 mg/kg on Day 1 and Day 15 of the 28-day cycle.

5. The method of claim 1, wherein administration of the anti-c-met antibody and the taxane is concurrent.

6. The method of claim 1, wherein administration of the anti-c-met antibody and the taxane is consecutive, in any order.

7. The method of claim 1, wherein administration of the anti-c-met antibody preceeds administration of the taxane.

8. The method of claim 1, 3, or 4, wherein the anti-c-met antibody is monovalent.

9. The method of claim 8, wherein the anti-c-met antibody comprises a single antigen binding arm and comprises a Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm.

10. The method of claim 8, wherein the anti-c-met antibody is an antibody or antibody fragment thereof, the antibody comprising (a) a first polypeptide comprising a heavy chain variable domain having the sequence:
EVQLVESGGGLVQPGGSLRLSCAASGYTFTSYWLHWVRQAPGKGLEWVGMIDP SNSDTRFNPNFKDRFTISADTSKNTAYLQMNSLRAEDTAVYYCATYRSYVTPLDYWGQ GTLVTVSS (SEQ ID NO:1), CH1 sequence, and a first Fc polypeptide; (b) a second polypeptide comprising a light chain variable domain having the sequence: DIQMTQSPSSLSASVGDRVTITCKSSQSLLYTSSQKNYLAWYQQKPGKAPKLLIYWAST R ESGVPSRFSGSGSGTDFTLTISSLQPEDFATYYCQQYYAYPWTFGQGTKVEIKR (SEQ ID NO:2), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

11. The method of claim 10, wherein the first polypeptide comprises the Fc sequence depicted in FIG. 1 (SEQ ID NO: 3) and the second polypeptide comprises the Fc sequence depicted in FIG. 2 (SEQ ID NO: 4).

12. The method of claim 1, 3, or 4, wherein the anti-c-met antibody binds the same epitope as onartuzumab.

13. The method of claim 1 3, or 4, wherein the anti-c-met antibody is humanized.

14. The method of claims 1 3, or 4, wherein the anti-c-met antibody is onartuzumab.

15. The method of claim 2 or 4, wherein said anti-VEGF antibody binds the same epitope as the monoclonal anti-VEGF antibody A4.6.1 produced by hybridoma ATCC HB 10709.

16. The method claim 15, wherein the anti-VEGF antibody is a humanized antibody.

17. The method of claim 16, wherein the anti-VEGF antibody is a humanized A4.6.1 antibody or fragment thereof.

18. The method of claim 16, wherein the anti-VEGF antibody is bevacizumab.

19. The method of claim 16, wherein the anti-VEGF antibody has a heavy chain variable region comprising the following amino acid sequence:
(SEQ ID NO: 31)
EVQLVESGGG LVQPGGSLRL SCAASGYTFT NYGMNWVRQA
PGKGLEWVGW INTYTGEPTY AADFKRRFTF SLDTSKSTAY
LQMNSLRAED TAVYYCAKYP HYYGSSHWYF DVWGQGTLVT
VSS
and a light chain variable region comprising the following amino acid sequence:
(SEQ ID NO: 32)
DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKR.

20. The method of claim 1, 3, or 4, wherein the taxane is paclitaxel.

21. The method of any one of the preceding claims, wherein the metastatic triple-negative breast cancer patient has been previously treated for metastatic triple-negative breast cancer patient.

22. The method of any one of claims 1-20, wherein the metastatic triple-negative breast cancer patient has not been previously treated for metastatic triple-negative breast cancer patient.

23. A method of promoting an anti-c-met antibody for the treatment of a metastatic triple-negative breast cancer patient, in combination with a taxane.

24. The method of claim 23, further in combination with an anti-VEGF antibody.

25. The method of claim 23, wherein the treatment comprises administering to a triple-negative metastatic breast cancer patient an anti-c-met antibody administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

26. The method of claim 24, wherein the treatment comprises administering to a triple-negative metastatic breast cancer patient an anti-c-met antibody administered at a dose of 10 mg/kg on Day 1 and Day 15 of a 28-day cycle, anti-VEGF antibody (e.g., bevacizumab) administered at a dose of 10 mg/kg on Day 1 and Day 15 of the 28-day cycle, and paclitaxel administered at a dose of 90 mg/m² by IV infusion on Day 1, Day 8, and Day 15 of the 28-day cycle.

27. The method of claim 23, wherein the promotion is by a package insert, wherein the package insert provides instructions to receive cancer treatment with an anti-c-met antibody.

28. The method of claim 23 wherein the promotion is by a package insert accompanying a commercial formulation of the anti-c-met antibody.

29. The method of claim 23, wherein the promotion is by a package insert accompanying a commercial formulation of the taxane.

30. The method of any one of claims 23 to 29, wherein the promotion is by written communication to a physician or health care provider.

31. The method of any one of claims 23 to 29, wherein the promotion is by oral communication to a physician or health care provider.

32. The method of any one of claims 23 to 29, wherein the promotion is followed by the treatment of the subject with the anti-c-met antibody or anti-c-met antibody.

33. A method of instructing a patient with triple-negative metastatic breast cancer by providing instructions to receive treatment with an anti-c-met antibody to increase survival of the patient, to decrease the patient's risk of cancer recurrence and/or to increase the patient's likelihood of survival.

34. The method of claim 33, wherein the treatment comprises administering to a triple-negative metastatic breast cancer patient an anti-c-met antibody administered at a dose of 10 mg/kg on Day 1 and Day 15 of a 28-day cycle, anti-VEGF antibody administered at a dose of 10 mg/kg on Day 1 and Day 15 of the 28-day cycle and paclitaxel administered at a dose of 90 mg/m² by IV infusion on Day 1, Day 8, and Day 15 of the 28-day cycle.

35. An article of manufacture comprising an anti-c-met antibody and/or a taxane, and a package insert or label with directions to treat a triple-negative metastatic breast cancer patient.

36. The article of manufacture of claim 35, further comprising an anti-VEGF antibody.

37. The article of manufacture of claim 35 or 36, wherein the taxane is paclitaxel.

38. The article of manufacture of any one of claims 35 to 37, wherein the treatment comprises administering to a triple-negative metastatic breast cancer patient an anti-c-met antibody administered at a dose of 10 mg/kg on Day 1 and Day 15 of a 28-day cycle, and paclitaxel administered at a dose of 90 mg/m² by IV infusion on Day 1, Day 8, and Day 15 of the 28-day cycle.

39. The article of manufacture of claim 36 or 38, wherein the treatment further comprises administering anti-VEGF antibody administered at a dose of 10 mg/kg on Day 1 and Day 15 of the 28-day cycle.

40. A method of manufacturing the article of manufacture of any one of claims 35 to 39.
